(19) United States
(12) Patent Application Publication
(10) Pub. No.: US 2026/0157743 A1
(43) Pub. Date: Jun. 11, 2026

(54) COMPOSITE WITH POROUS SWELLING COPOLYMER FOR BONE FIXATION

(71) Applicant: Drexel University, Philiadelphia, PA (US)

(72) Inventors: Sorin Siegler, Philadelphia, PA (US); Giuseppe R. Palmese, Hainesport, NJ (US)

(73) Assignee: Drexel University, Philadelphia, PA (US)

(21) Appl. No.: 18/821,187

(22) PCT Filed: Mar. 2, 2023
(86) PCT No.: PCT/US2023/063546

§ 371 Date: Aug. 30, 2024

Related U.S. Application Data
(60) Provisional application No. 63/315,833, filed on Mar. 2, 2022.

(51) Int. Cl. A61B 17/04; A61B 17/00

(52) U.S. Cl. CPC A61B 17/0401; A61B 2017/00898; A61B 2017/044

(57) ABSTRACT

The present invention relates to an implantable device that may be used for bone fixation comprising a combination of a solid swelling copolymer and a porous swelling copolymer. The present invention also relates embodiments where the implantable device is a suture anchor, and to osteochondral defect repair systems comprising the implantable device. The anchor may be configured for anchoring a repair system to bone; and a joint interface component can be affixed to the anchoring component and may be provided with a smooth surface adapted for articulation of natural cartilage of opposing bone along said surface.

### CROSS REFERENCE TO RELATED APPLICATIONS

This application claims the benefit of U.S. Provisional Application No. 63/315,833, filed on Mar. 2, 2022, the entire disclosure of which is hereby incorporated by reference as if set forth fully herein.

### BACKGROUND

In modern orthopedics, fixation of artificial or natural components to bone has always presented major challenges. Several factors contributed to, and exacerbated, the problem including anatomical sites with low density bone such as the humeral head at the shoulder, presence of osteoporosis, stress shielding by an anchor with much higher stiffness than the surrounding bone, and lack of osteointegration.

Fixation to bone using bone anchors is required in many orthopedic applications and surgical procedures such as reattachment of torn soft tissue to bone, fixation of plates to treat complex fractures, fusion of bones whose joint surfaces may have been affected by arthritis, performing various osteotomies, using implants (autographs, allographs and artificial) to repair osteochondral defects, internal fracture fixation [11-14], soft tissue reattachment [15-18], soft tissue reconstructions [19-21], deformity corrections [22, 23], repair of osteochondral defects [24], fusions, and various osteotomies [25, 26]. Currently fixation is achieved using screws, anchors, pegs, or bone grafts of artificial material (metal, polymers, polyether ketone (PEEK)) or natural material such as bone/cartilage plugs from human cadavers or the patient. Their main function is to provide stable long-term fixation to bone.

Conventional bone fixation systems have some inherent problems. One such problem is poor fixation strength particularly in cancellous bone, and especially when such bones have low density. The attachment sites of the rotator cuff tendon in the shoulder are known for their poor fixation strength. Similarly, osteoporosis may significantly reduce bone density resulting in poor fixation strength.

Another problem is stress shielding resulting from a mismatch between the high modulus of elasticity of the conventional bone anchor and the relatively low modulus of elasticity of bone. Such a mismatch may result in bone resorption and weakening of the bone in the vicinity of the fixation, leading to failure.

Another problem with conventional bone anchors is a lack of osteointegration. For good osteointegration a minimum amount of porosity is required in the implant so that bone can grow into the implant and form a tight, intimate bond with the implant. In the case of repair systems for osteochondral defects, low bone density often results in subsidence of the implant into the bone due to contact forces acting on the implant thereby compressing it into the low-density bone. Once that happens, the implant fails to provide smooth motion at the joint and pain and high friction develops leading to failure and a need for surgical repair. These inherent deficiencies give rise to surgery-related failures produced by anchor pullout, graft subsidence, and implant loosening.

Most metallic commercial bone anchors, such as screws and suture anchors achieve their fixation to bone through shear of the bone located between the threads. [27, 28]. Their pullout strength is also achieved through shear of the bone located between the threads [28-20]. They have several deficiencies, potentially leading to failure, which are particularly evident in low density bone. These include stress-shielding resulting from mechanical properties mismatch; lack of mechanically induced remodeling and osteointegration; and when the pull-out force on the anchor, during functional activities, exceeds the pull-out strength, catastrophic failure occurs leaving behind large bone defects that may be hard to repair.

Traditionally, these conventional anchors were made of metal such as stainless steel and titanium with a modulus of elasticity several times higher than that of bone [31, 32]. This mismatch in moduli may create stress-shielding inducing bone resorption which may lead to long-term loosening of the anchor [33, 34]. Recently, other materials such as PEEK and resorbable polymers were introduced with moduli of elasticity closer to that of bone, reducing stress-shielding effects [35, 36]. However, these anchors, similar to the metal ones, rely on a shear resistance mechanism for their fixation, and thus, result in poor fixation and pull-out stress in low density bone [37-43]. Such low-density bone occurs at certain anatomical sites such as the shoulder, or in osteoporotic bone [44]. These inherent deficiencies give rise to implant failures such as anchor pullout, graft subsidence, and implant loosening. When the force acting on the bone anchor exceeds its pull-out strength, the bone anchor completely loses its fixation capacity, and when pulled-out of the bone, may leave behind a large defect [45, 46].

Therefore, improving the quality of current bone fixation techniques by addressing these factors, i.e. optimizing fixation in low-density bone, preventing bone resorption, and promoting osteointegration, may significantly improve the outcome of many orthopedic surgeries.

To address some of the problems associated with conventional bone anchors a new type of bone anchor was introduced in the past [47-50]. These relatively new bone anchors are made of a copolymer with one component being hydrophobic and the other hydrophilic [47, 48]. The ratio between these polymers can be used to control the amount of swelling of the anchor. Bone anchors made of these materials achieve their fixation to bone by absorbing interstitial fluids and swelling by a controlled amount, thus producing radial stresses in the surrounding bone [50-53]. These radial stresses result in frictional resistance against pull-out forces which are acting on the anchor during functional activities.

Bone anchors made of the co-polymeric swelling material have some significant advantages over conventional threaded anchors. As described earlier, while the conventional threaded anchors achieve their fixation strength through a shear-based mechanism, the swelling anchors achieve their fixation strength via a friction-based mechanism [50-52]. Therefore, it is expected that in lower density bone, the relative fixation stress of the swelling anchors compared to conventional threaded anchors increases, making them more desirable for low density bone such as for suture anchors for rotator cuff repair or for fixing suture anchors in osteoporotic bone. In addition, due to the difference in the fixation mechanism (friction vs. shear), the swelling anchors possess a re-fixation capability in contrast to conventional anchors that become loose once the maximum pull-out force has been reached.

Osteointegration of an orthopedic implant represents the process by which, through bone remodeling, bone cells migrate into the porous structure at the bone-implant interface resulting in an intimate fixation [56-58]. Osteointegration is a highly desirable phenomenon since it creates a mechanically strong construct, resulting in long-term fixation and thus mitigating some of the reasons for implant loosening. Consequently, many modern orthopedic implants, such as the stems of i.e., uncemented total joint replacement components, possess a porous surface allowing for osteointegration [56-62]. Since bone remodeling and osteoconductive properties are essential for osteointegration into the pores of a biomaterial, surface derivatization using various biological agents and inorganic elements can additionally enhance the process by creating an osteoconductive milieu. These include anti-osteoporosis drugs such as Strontium ranelate and simvastatin [63-67], bioactive molecules such as bone morphogenetic protein (BMP) and vascular endothelial growth factor (VEGF) [58, 68-70], and bioactive inorganic elements such as calcium (Ca), strontium (Sr), magnesium (Mg), zinc (Zn), and silicon (Si) [71-73]. Mechanotransduction is a critical determinant of new bone formation, repair and regeneration, and adaptation of bone to its external environment. This response of bone to mechanical stimulus is known as Wolff's law [74-78] and various forms of mechanical stimulus that promotes bone remodeling has been extensively investigated in the past [78]. However, few, if any, present-day commercial implants can produce radial stresses in the bone-implant interface to mechanically induce controlled bone remodeling.

### SUMMARY OF THE INVENTION

To overcome the deficiencies in current implantable devices, the present invention demonstrates the swelling and compressive mechanical characteristics of a porous swelling co-polymeric material as an implantable device. Porosity may be provided by adding a dissolvable agent, such as sodium chloride (NaCl), during the process of polymerization of the co-polymeric material. The agent is later leached out of the polymer by, for example, dissolving it in water, leaving behind a porous structure with adequate porosity for osteointegration.

Three different groups of samples of the swelling co-polymer are described herein, 1) a solid cylindrical sample, 2) a fully porous cylindrical sample, and 3) a partially porous cylindrical sample with a solid core and a porous outer layer. The partially porous swelling co-polymer of the present invention demonstrated excellent mechanical properties matching those of cancellous bone, a quick swelling response, and included an adequate porous outer layer for mechanically induced osteointegration in swelling and simple compression studies. These results indicate that this partially porous material may provide an effective alternative to conventional bone anchors, particularly, in low density bone.

The present invention relates to several bone fixation products that improve the outcome of orthopedic surgery by using a porous co-polymer with controlled fluid-induced expansion properties as part of the bone-fixation mechanism. Such products will improve surgical outcomes by increasing fixation strength to bone, preventing bone resorption, and promoting osteointegration. This will impact thousands of patients undergoing orthopedic procedures that require bone fixation in procedures, for example, but not limited to, rotator cuff repairs, ligament repairs, tendon transfers, and repair of osteochondral defects.

In one aspect, the invention is directed to suture anchors for soft tissue reattachment in the extremities such as rotator cuff repairs of the shoulder.

In another aspect, the invention relates to artificial or natural grafts for repair of osteochondral defects including surgical management of Hallux Rigidus (1, 2), and repair of osteochondral defects of the talar dome (3, 4).

In another aspect, the invention is directed to a device for bone fixation including a combination of a solid swelling copolymer and a porous swelling copolymer. In one embodiment of this aspect, the device includes an inner core of a solid swelling copolymer and an outer layer of a porous swelling copolymer surrounding the inner core.

In another embodiment of this aspect, the porous swelling copolymer may have an average pore size of from about 50 microns to about 750 microns, or from about 250 microns to about 700 microns, or from about 400 microns to about 600 microns.

In each of the foregoing embodiments of the bone fixation device the solid swelling copolymer and porous swelling copolymer may be comprised of the same copolymer.

In each of the foregoing embodiments of the bone fixation device the porous swelling copolymer may have a porosity of from about 30% to about 70%, or from about 40% to about 60%, or about 50%.

In each of the foregoing embodiments of the bone fixation device the solid swelling copolymer and the porous swelling copolymer may comprise a crosslinked copolymer of a hydrophobic monomer and a hydrophilic monomer.

In each of the foregoing embodiments of the bone fixation device the hydrophobic monomer may be methyl methacrylate and the hydrophilic monomer may be acrylic acid.

In each of the foregoing embodiments of the bone fixation device the copolymer(s) may be crosslinked using allyl methacrylate.

In a further aspect, the present invention relates to a suture anchor including a tip, an anchoring element including a distal portion configured for connection to the tip, a proximal portion provided with circumferential threads on an outer surface thereof and including two suture tunnels therein each of which are adapted for receiving a suture, and a central portion between the distal and proximal portions including an additional suture tunnel adapted for receiving the suture; a hollow swelling sleeve including a tube of a solid swelling copolymer configured for surrounding the central portion of the anchoring element, and an outer layer of a porous swelling copolymer surrounding the tube.

In a still further aspect, the present invention relates to a suture anchor including a cylindrical anchor element of swelling copolymer having a tip on a distal end thereof and threads on a portion of an outer surface thereof and which is adapted for securing a suture.

The suture anchor may be paired with a suture. In this embodiment of the suture anchor the suture may be secured to the anchor element during copolymerization of the anchor element.

In each of the foregoing embodiments of the suture anchor the anchor element may include an internal tube for receiving the suture, the internal tube having both an inlet and an outlet on a proximal end of the anchor element.

In yet another embodiment the invention relates to an osteochondral defect repair system including an anchoring element comprising a swellable copolymer configured for anchoring the repair system to bone; and a joint interface component affixed to the anchoring component and having a smooth surface adapted for articulation of natural cartilage of opposing bone along said smooth surface.

The osteochondral defect repair system may have a joint interface component including a polyvinyl alcohol cryogel, a chemically crosslinked hydrogel, or a natural allograft or autograft.

The osteochondral defect repair system of each of the foregoing embodiments may have an anchoring element with a controlled variable porosity.

In the osteochondral defect repair system of each of the foregoing embodiments, the anchoring element may include a threaded outer surface portion.

### BRIEF DESCRIPTION OF THE DRAWINGS

FIG. 1 shows swelling and mechanical characterization of solid samples of the swelling co-polymer confirming the ability to produce a swelling implant with the desired swelling and mechanical properties.

FIG. 2 shows the process of producing the porous swelling co-polymeric samples using sacrificial template particles, such as NaCl crystals. NaCl crystals are added to the resin prior to curing. Following curing, NaCl crystals are dissolved in 0.9% distilled water leaving behind a porous structure of the swelling co-polymer.

FIG. 3 shows the production of porous copolymeric swelling components using stereolithographic Digital Light Processing (DLP) printing.

FIG. 4 shows a device according to the present invention comprising a solid inner core and a porous swelling outer layer.

FIG. 5, including sections 5A and 5B and photos 5C and 5D, show a knotless swelling suture anchor design and prototype for applications such as rotator cuff repair, according to embodiment 1. Sections 5A and 5B of FIG. 5 are cross-sectional views taken along a vertical plane through the center of the device.

FIG. 6 shows a cross sectional view of a suture anchor according to embodiment 2. The suture anchor of FIG. 6 comprises a titanium or shape-memory alloy. The core section, represented by a crisscross pattern may be a perforated surface which allows for body fluid and bone ingrowth. The sleeve surrounding the perforated surface is the swelling copolymer according to the present invention.

FIG. 7 shows a swelling suture anchor according to embodiment 3. The suture anchor of FIG. 7 comprises a swelling copolymer exterior comprising a sharp tip and threads to assist in fixation of the suture anchor to a bone. The suture anchor comprises a protective U-shaped tube into which a suture may be inserted or the suture may be fixed within the tube.

FIG. 8 shows an osteochondral defect repair system comprising an “artificial cartilage” polyvinyl alcohol (PVA) first part attached to a porous swelling copolymer anchor second part.

FIG. 9 shows compression mechanical testing of PVA and an embodiment of the PVA/swelling copolymer system.

FIG. 10 shows a second embodiment of the osteochondral defect repair system. As shown in FIG. 10, the repair system includes a functional component for a joint interface 3, a controlled swelling copolymer 2, and a swelling copolymer with controlled variable porosity 1. The functional component for a joint interface 3 that resembles artificial cartilage may comprise a polyvinyl alcohol (PVA), cryogel, or a chemically crosslinked hydrogel. The controlled swelling copolymer 2 provides controlled swelling along the length of the implant to match the increased porosity of the bone. The swelling copolymer with controlled variable porosity is the swelling copolymer of the present invention.

FIG. 11 shows cross-sectional views of the three different sample types (solid (11A & 11B), porous (11C & 11D), and hybrid (11E & 11F) and the corresponding EMS images of their microstructure. 11A and 11B show solid swelling co-polymer. 11C and 11D show a porous swelling co-polymer. 11E and 11F show a hybrid swelling co-polymer.

FIG. 12 shows an example of the stress vs. strain behavior of a swelling sample tested in simple compression. The sample graph shows the definition for the elastic modulus—E, the ultimate stress and ultimate strain—σ_{y} and ε_{y}. Toughness is defined as the area under the stress-strain curve to a strain of 0.3.

FIG. 13 shows orthopedic surgical devices with controlled fluid-induced expansion properties for improved bone fixation and bone integration.

FIG. 14 shows problems associated with poor fixation in low density bones.

FIG. 15A shows the material production of a hybrid structure comprising a solid core with a porous outer layer.

FIG. 15B shows the compressive strength and modulus of elasticity of various materials.

FIG. 16 shows the pull-out test setup for testing the suture anchors according to the present invention and prior art.

FIG. 17A shows the refixation properties of a swelling anchor having a bone density of 8 pounds per cubic foot (PCF 8).

FIG. 17B shows the refixation properties of a swelling anchor having a bone density of 15 pounds per cubic foot (PCF 15).

FIG. 17C shows the refixation properties of a swelling anchor having a bone density of 30 pounds per cubic foot (PCF 30). FIG. 18 shows that autoclave sterilization does not affect swelling or mechanical properties of the implants.

FIG. 19 shows an in vitro cell study. In FIG. 19a, bone marrow was taken from the tuber coxae of a standing skeletally mature sheep. Mesenchymal Stem Cells (MSC) were isolated from the bone marrow. In FIG. 19b, osteoblast cells were produced and proliferated for the biocompatibility evaluation. In FIG. 19c, the images compare the viability of cells on a substrate formed from the swelling co-polymer and cells on a standard substrate used for this purpose.

FIG. 20A shows the setup for carrying out a pull-out test for the in vitro study in sheep.

FIG. 20B shows the results from the pull-out test using a non-swelling anchor in a lumbar spine of a sheep.

FIG. 20C shows the results from the pull-out test using a non-swelling anchor in a femur of a sheep.

FIG. 20D shows the results from the pull-out test using a non-swelling anchor in a lumbar spine of a sheep. (Different from FIG. 20B).

FIG. 20E shows the results from the pull-out test using a swelling anchor in a lumbar spine of a sheep.

FIG. 20F shows the results from the pull-out test using a swelling anchor in a lumbar spine of a sheep.

FIG. 20G shows the results from the pull-out test using a swelling anchor in a lumbar spine of a sheet.

FIG. 21 shows a Digital Light Processing 3D printing technique and UV photo-initiator that works with acrylic acid (AA), methyl methacrylate (MMA), and diethylene glycol dimethacrylate (DEGDMA). FIG. 21 shows a method of creating a porous swelling structure according to the present invention using a 3D printing approach which may be an alternative approach to the method of creating porosity using a water soluble crystal, such as salt.

### DETAILED DESCRIPTION

The viability and improved performance of the products of the invention are demonstrated by surgical-expert evaluation, biomechanical testing on artificial and natural bone, and in vivo animal implantation studies.

Immediate beneficiaries of these products may include patients with severe soft tissue tears and avulsions such as a rotator cuff tear, an Achilles' tendon rupture, an ankle ligament tear, and various injuries involving tendons located in the extremities.

Another patient group that stands to benefit from such products includes those with severe pain and limited motion due to arthritis of the big toe (Hallux Rigidus) or osteochondral lesions resulting from trauma. For this group, relief is achieved by artificial or natural bone/cartilage grafts, which often fail due to subsidence of the graft into the soft cancellous bone. Patients in these groups are treated by orthopedic surgeons who have become aware of the difficulties and high failure rates of current soft tissue reattachment procedures, particularly in regions of low-density bone such as the shoulder or in osteoporotic bone. These surgeons have also noted difficulties and high failure rates of osteochondral defect repair systems (CARTIVA™ (6) being a recent example).

Adoption of the products of the present invention by these surgeons and their patients, will be driven by the technical, biological, and clinical evidence of improved fixation strength, excellent osteointegration, and outstanding biocompatibility, resulting in reduced surgical failures and a lesser need for revision surgeries, thereby positively impacting patient quality of life. In addition, insurance companies that bear the cost of these surgeries will benefit from a reduction in costs associated with reduced surgical failures. As such, it is anticipated that the products of the invention will not increase the usage cost, when compared to existing, less effective solutions.

The envisioned products will achieve these goals due to their self-fixation properties that lead to an increased bone incorporation strength over existing products, eliminate bone resorption and promote bone integration.

The invention addresses the deficiencies of conventional bone anchoring systems. The products include a swelling co-polymer that may be porous, either by itself or in combination with components made of other material(s) such as metals (titanium, stainless steel), polyether ketone (PEEK), polyvinyl alcohol cryogels, or other chemically crosslinked hydrogels.

The swelling co-polymer is made from a combination of a hydrophobic monomer, such as methyl methacrylate, and a hydrophilic monomer, such as acrylic acid, using a crosslinking agent such as diethylene glycol di(meth)acrylate (DEGDMA) or allyl (meth)acrylate. By controlling the ratio of the hydrophilic to hydrophobic monomers the degree of swelling can be controlled. Fixation to bone is obtained as the copolymer absorbs body fluids in situ and swells or attempts to swell, thereby applying pressure on surrounding bone in which the swellable copolymer is confined. When contacted with fluids, the swelling copolymer applies radial stresses on the bone producing effective frictional forces that fix the implant in the bone thereby providing strong resistance to pull-out or push-in forces that may be exerted on the implant.

The swelling copolymer may itself form an entire bone anchor or implant, the swellable copolymer may be employed in combination with other materials such as titanium, stainless steel, PEEK, polyvinyl alcohol cryogels, chemically crosslinked hydrogels, etc. These components may together provide the desired function such as anchoring soft tissue to the bone, providing a smooth surface for joint motion in osteochondral repair, attaching fracture fixation plates to bone, etc.

In addition, the swelling produces radial stresses in the bone which will cause new bone formed after implantation to infiltrate into pores in the swelling material. For this purpose, the swelling copolymer can be fabricated with a controlled porosity. This will enhance osteointegration of the implant further improving the fixation strength of the implant to the surrounding bone.

The material properties of the swelling copolymer can be tailored to be closer to the properties of bone than metals or non-metallic materials such as PEEK, to thereby reduce or prevent bone resorption to ensure long-term effective fixation and minimize the need for surgical repair.

The swelling copolymer component may be a composite material containing a solid swelling core element and a porous swelling outer element. Such a porous element is preferably located between the solid swelling core element and the bone into which the component is to be inserted to allow for osteointegration of new bone growth. The porous element provides for ingrowth of bone into the pores of the component and also allows for migration of fluids from the body to the swelling core element to cause the core to swell or at least exert internal pressure on the surrounding porous element. The combined forces resulting from absorption of fluids by both the core element and the porous element help to hold the system in place within the bone while the bone grows into the pores of the porous element to provide the final long-term bone fixation.

The pore size, distribution and porosity of the porous swelling copolymer can be customized for the specific end use of the final structure. For example, the number and/or size of the pores may increase as the depth into the bone increases. Not to be bound by theory, but larger pore sizes and greater porosity may allow for enhanced fluid penetration to the solid swelling core, which can provide faster initial swelling of the device, to thereby promote bone ingrowth by increasing pressure on the surrounding bone.

For example, the bone fixation device may have different porosities at different locations and the porosity of the porous swelling copolymer may also vary at different locations in the device. The porous swelling copolymer of the bone fixation device may have a porosity that is greater at an outer portion of the porous swelling copolymer to less at an inner portion of the porous swelling copolymer. For example, the outer portion of the porous swelling copolymer of the bone fixation device may have a pore size of from about 50 microns to about 750 microns, or from about 50 microns to about 675 microns, or from about 100 microns to about 600, and the inner portion of the swelling copolymer may have a pore size of from about 50 microns to about 700 microns, or from about 100 microns to about 750, or from about 250 microns to about 750 microns so long as the porosity of the inner portion of the porous swelling copolymer is less than the porosity of the outer portion of the porous swelling copolymer. The outer portion of the porous swelling copolymer may have a porosity of from about 30% to about 70%, and the inner portion of the porous swelling copolymer may have a significantly lower porosity than the outer portion of the bone fixation device, preferably from 10% to 50% or 20% to 40% or 30% to 40%.

Preferably, the average pore size of the swellable copolymer can range from about 50 microns to about 750 microns, and, more preferably, the average pore size can range from about 250 microns to about 675 microns, and, most preferably, the average pore size can be from about 400 microns to 600 microns, or about 500 microns, which has been shown to be an average pore size that is suitable to support the desired amount of bone ingrowth.

The porosity of the porous swelling copolymer is preferably in a range of about 30% to about 70%, more preferably, from about 40% to about 60% and, most preferably, about 50%.

Suitable examples of hydrophilic monomers that can be employed in the copolymerization process include 2-hydroxy ethyl (meth)acrylate (HEMA or HEA), N,N-dimethyl acrylamide, N-Isopropylacrylamide (NiPAM), 2-dimethylaminoethyl (meth)acrylate (DMAEMA), dimethylaminoethyl acrylate DMAEA), acrylic acid (AA), polyethylene glycol (meth)acrylate (PEG-MA or PEG-A) and glyceryl mono(meth)acrylate.

Suitable examples of hydrophobic monomers that can be employed in the copolymerization process include styrene, vinyl toluene, carvacryl (meth)acrylate, menthyl (meth)acrylate, thymyl (meth)acrylate, cyclohexyl (meth)acrylate, butyl (meth)acrylate and hexyl (meth)acrylate.

Suitable hydrophobic crosslinkers for use in the copolymerization process may include hexane diol di(meth)acrylate, butane diol di(meth)acrylate, and bisphenol A-glycidyl methacrylate (BisGMA). Suitable hydrophilic crosslinkers for use in the copolymerization process may include ethylene glycol di(meth)acrylate and polyethylene glycol di(meth)acrylate.

The solid swelling copolymer may be present in an amount of 50-95 wt. % of the total copolymer content of the component and the porous swelling copolymer may be present in an amount of 5-50 wt. % of the total copolymer content of the component.

A group of swelling crosslinked copolymeric materials capable of self-fixation to bone were designed, produced and tested. The crosslinked copolymers consisted of copolymers of methyl methacrylate (MMA) and acrylic acid (AA). After characterizing the mechanical and swelling properties of these materials, their self-fixation characteristics were tested under a variety of conditions (5, 6). For example, pull-out and push-out tests were performed in foam substrates of different densities that were fabricated with material properties and structures resembling those of cancellous bone (7). These tests were repeated in vitro in bovine bone samples, and lastly in vivo testing was conducted on fifteen white New-Zealand rabbits (8).

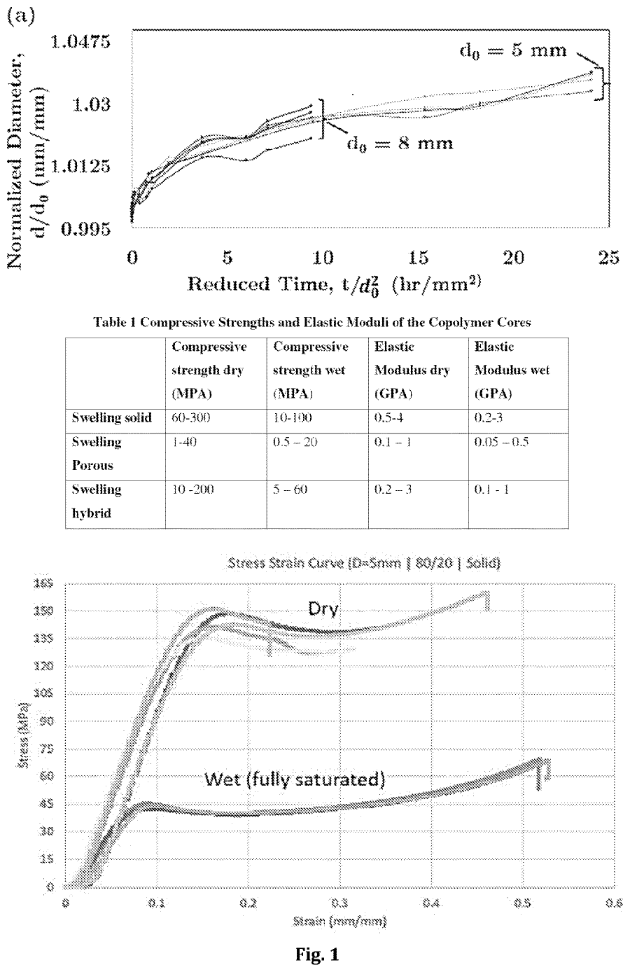

Essentially the same crosslinked copolymers were later employed to successfully reproduce the results discussed above. This was confirmed by comparing the swelling and mechanical characteristics of the reproduced crosslinked copolymers and very similar results were obtained as shown in FIG. 1. Specifically, most of the swelling occurred within the first 24 hours with the copolymer structures swelling to a final diameter increase of about 6%. The dry compressive strength was found to be 120 MPa and was reduced to 47 MPa when the crosslinked copolymer was fully saturated. The dry modulus of 1.7 GPa was reduced to 0.9 GPa upon reaching full saturation. The material became more ductile as it swelled.

The amount of swelling of the copolymer may be from about 1% to about 25% of either the original diameter or the original volume, more preferably, about 3% to about 20% and most preferably about 5% to about 15% of the original diameter or the original volume. These ranges apply both to the solid swellable core and the porous part of the component.

To produce swelling porous copolymers, dissolvable particles such as salt particles of various sizes, were employed as sacrificial template particles. The composite materials were comprised of an AA-MMA copolymer matrix and closely-packed NaCl crystals. The salt crystals were then leached out of the composite by washing with water and drying to form the porous swellable structure (FIG. 2). Using this method, porous swelling copolymers with varying percentages of porosity and different pore sizes can be produced. Exemplary copolymers had pore sizes of about 500 microns, and copolymers with this pore size were shown to promote osteointegration.

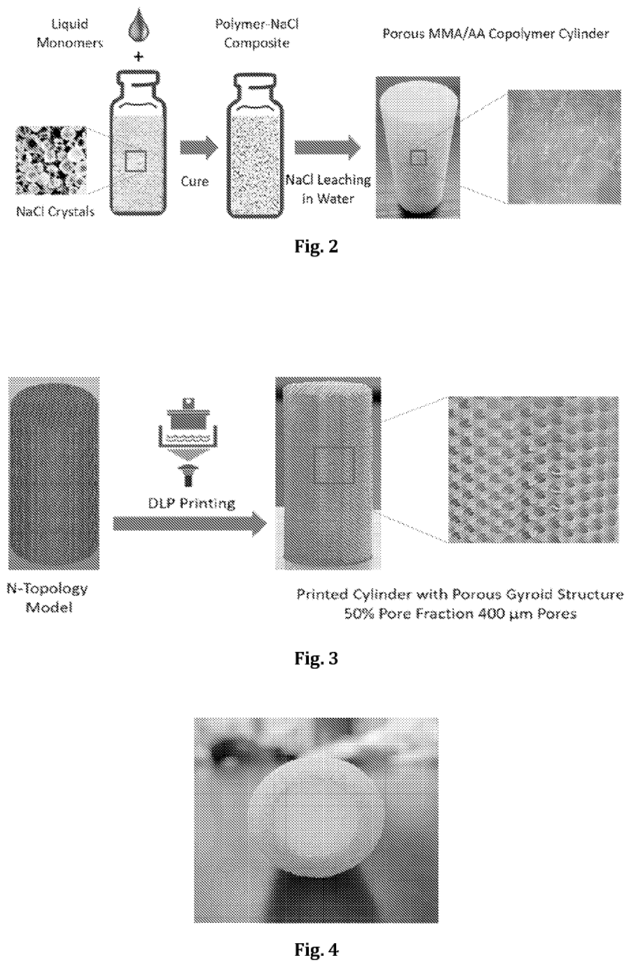

An alternative method for producing the desired pores of various shapes and sizes is a 3D Stereolithographic Digital Light Processing (DLP) technique with a resolution of 50 microns. An example of a porous structure that was made using this technique is shown in FIG. 3. Using this technique with the AA-MMA swelling copolymer requires the selection of a suitable combination of conventional amounts of a known photo-initiator and/or a known photo-absorber.

Stereolithographic DLP printing allows for the creation of porous polymeric structures with controlled pore properties including pore size, % porosity and pore size distribution. The swelling and mechanical characteristics of the porous copolymers made by this process were characterized. The porous structures reached maximum swelling in less than 5 hours. The dry compressive strength was 9.4 MPa which was reduced to 3.4 MPa when fully saturated. The dry modulus of 0.25 GPa was reduced to 0.14 GPa upon reaching full saturation. Compared to the solid structure, the porous structure reached saturation much faster, was weaker, was less stiff and remained ductile.

For some applications only a partial porosity may be desired in which case different combinations of solid swelling copolymer with porous swelling copolymer are envisioned. One preferred swelling structure includes an inner, solid swellable core provided with a porous outer layer that forms a shell around the inner core. The inner core provides most of the swelling and strength to the device while the porous outer layer promotes osteointegration and allows fluid penetration to the inner core.

In addition to providing room for bone ingrowth the porous structure increases surface area so that the characteristic swelling time of the porous layer is much faster compared to the solid core. This provides a fast initial fixation that then strengthens as the core swells more slowly. The swelling plot in the FIG. 1 can be used to estimate the time of swelling of the porous structure based on the characteristic size of the porous structure. The plot is made in reduced time and two sets of swelling data for different sized cylinders are superimposed therein. Assuming a characteristic thickness of the polymer in the porous structure to be 0.1 mm, then the swelling to saturation of the porous material would be on the order of 6 hours. By comparison it takes 600 hours for swelling of the 5 mm cylinder. Adding the porous layer also reduces the time for the core to swell because it effectively provides a solid cylinder with a significantly reduced diameter. The 8 mm cylinder takes about 1600 hours to reach saturation (t/d²=25).

A two-stage polymerization process was employed to fabricate this core-shell structure, starting with a first polymerization stage to form the solid swellable core, followed by a second polymerization stage to form the porous outer layer. An exemplary core-shell device is shown in cross-section in FIG. 4.

The swelling and mechanical properties of the core-shell swelling device were evaluated. A saturation swelling ratio slightly below that of a completely solid structure was obtained. The dry compressive strength was 59.7 MPa and the dry modulus of elasticity was 0.7 GPa.

### EMBODIMENTS

The porous, swelling copolymers can be used either alone or in combination with other materials as an implantable device to, for example, provide an efficient fixation to bone for use in a significant number of different orthopedic applications. The device may be in the form of devices such as suture anchors, screws and pegs of various sizes, shapes, and forms, interference screws and osteochondral defect repair systems. The present exemplified embodiments are focused on two major applications where strong fixation is vital but challenging. The first is suture anchors for soft tissue reattachment in the extremities such as rotator cuff repairs at the shoulder. The second is grafts for repair of osteochondral defects including surgical management of Hallux Rigidus (arthritis of the MTP joint of the first ray), and repair of osteochondral defects.

### Suture Anchors

Suture anchors are used to reattach torn tendon or ligament to bone. They consist of the anchor and a suture attached to the anchor. In the reattachment procedure, the suture is passed through the torn ligament or tendon and tightened back to the bone.

The embodiment 1 shown in FIG. 5 includes a tip that may be made of a hard material such as metal or PEEK, an anchor element that may be made of a material such as metal or PEEK and a tubular swelling sleeve. As shown, these three components of the suture anchor are separable elements to allow association of the suture anchor with a suture, as shown in FIG. 5, prior to assembly of the suture anchor. More specifically, the suture is passed through the three tunnels in the anchor element that are adapted for receiving the suture as shown in FIG. 5A. The suture anchor may then be assembled by passing the central and distal portions of the anchor element through the tubular swelling sleeve and screwing the distal portion of the anchor element into a recess provided in the proximal end of the tip as shown in FIG. 5B. In this manner, the suture may be trapped at a fixed position between the anchor element and the tubular swelling sleeve as shown.

The tip of the suture anchor may be provided with a sharp point at its distal end and has for its primary function to facilitate penetration of the suture anchor into bone and/or other hard tissue. A second function of the tip is to provide an attachment point for the anchor element in the form of a cylindrical threaded recess in the proximal end of the tip. The threaded recess is sized to receive and mate with the outer threads on the distal portion of the anchor element to allow fixation of the anchor element to the tip by screwing the distal portion of the anchor element into the recess in the proximal end of the tip.

The anchor element includes a cylindrical distal portion provided with threads on the outer surface thereof. The cylindrical distal portion is sized to fit into the recess in the proximal end of the tip and the outer threads of the distal portion of the anchor element mate with the inner threads of the recess in the proximal end of the tip to provide a threaded connection between the anchor element and the tip. A suitable material for the threads is titanium.

The anchor element also includes a cylindrical proximal portion provided with threads on its outer surface and a central portion that connects the distal and proximal portions of the anchor element. The threads on the outer surface of the proximal portion are provided for the initial fixation of the suture anchor to the bone during the surgical procedure by screwing the suture anchor into the bone such that these threads engage the bone to fix the suture anchor in position. As such, the outer circumference of the proximal portion of the suture anchor must be of sufficient size that the threads on the outer surface extend beyond the outer diameter of the remainder of the suture anchor as shown in FIG. 5B. The proximal portion also forms a cylindrical shoulder at its distal end that rests on a proximal surface of the tubular swelling sleeve when the suture anchor is assembled such that the suture is pinched between this cylindrical shoulder and the proximal surface of the tubular swelling sleeve as shown in FIG. 5B.

The proximal portion of the anchor element is provided with two suture tunnels as shown so that a suture may be passed into the suture anchor via one of these two suture tunnels and out of the suture anchor via the other of these two suture tunnels, as shown in FIG. 5A.

The central portion of the anchor element is sized to fit within the tubular swelling sleeve as shown in FIG. 5B. A third suture tunnel is provided in the central portion to allow the suture to pass through the central portion between passing through the first and second suture tunnels of the proximal portion as shown in FIG. 5A.

The tubular swelling sleeve is also cylindrical, and it is configured to fit around the central portion of the anchor element. The tubular swelling sleeve includes a solid tube of the swelling copolymer and an outer tubular layer of porous swelling copolymer.

The suture anchor is sized to locate the threads on the outer surface of the proximal portion proximate to the surface of the bone to provide immediate fixation of the suture anchor to the bone. The immediate fixation is subsequently augmented by additional fixation provided by pressure exerted as a result of swelling of the swelling sleeve, as well as osteointegration of new bone into the pores of the outer porous tubular layer of the tubular swelling sleeve.

Embodiment 1 of the suture anchor is designed for repair of the rotator cuff. However, similar designs with varying dimensions and optimized for specific anatomical sites can be provided for other types of procedures.

The suture anchors of embodiment 1 were inserted into artificial bone and allowed to swell in saline solution for 10 days. Push-out tests conducted on swelled samples measured push-out forces of 705 N. Scaling this force by the cross-sectional area of the sample would provide a maximum average pull-out stress of about 0.82 N/mm². In comparison, the pull-out stress of popular suture anchors used in rotator cuff repair (such as the 5.5 mm Corkscrew by Arthrex™) has been reported as varying between 0.25 N/mm² to 0.77 N/mm². As such, the suture anchor of embodiment 1 is predicted to provide a higher pull out stress than this commercially available suture anchor. Higher pull-out stress is desired to ensure long-term, stable positioning of the suture anchor.

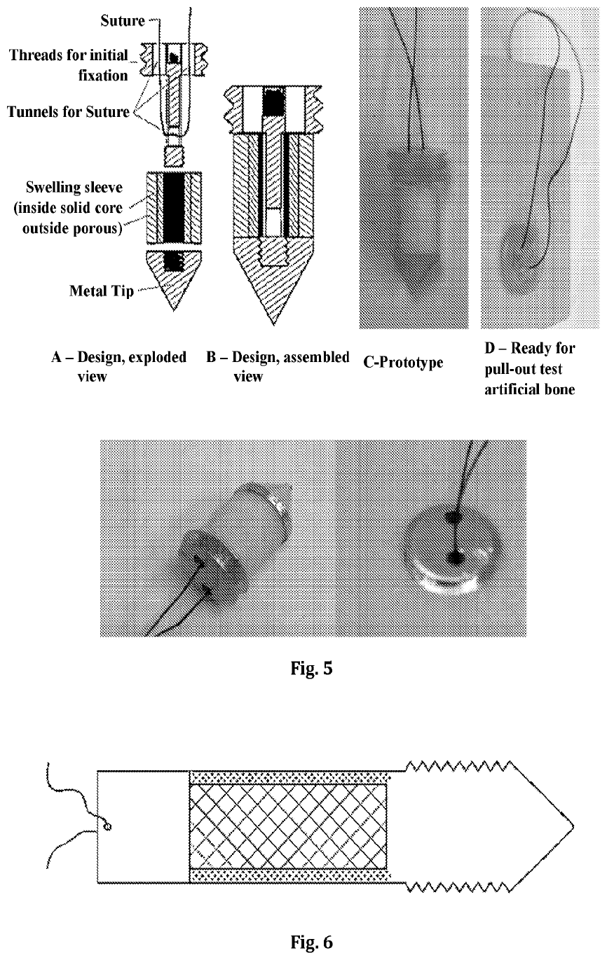

Another embodiment, embodiment 2 of a suture anchor is shown in FIG. 6.

Embodiment 2 of the suture anchor includes an anchor element and a swelling element. The anchor element may be made of titanium, PEEK, stainless steel, a shape memory alloy or another similar material and is designed to provide the initial fixation of the suture anchor to the bone upon implantation using the threads located on an outer surface of the anchor element.

The anchor element of the suture anchor of embodiment 2 is partially hollow to provide an internal cavity and an expandable wall surrounding the internal cavity is thin and perforated to allow fluid penetration into the cavity and expansion of the wall under internal pressure. In one embodiment, the expandable wall may be in the form of a mesh made of a material that is sufficiently flexible to expand upon exertion of pressure on the internal surface of the wall by swelling of a swellable material located in the internal cavity. The anchor element also includes a sharp tip to facilitate insertion of the suture anchor into the bone.

The internal cavity is filled with the swelling copolymer such that when body fluids reach the swelling copolymer the copolymer swells and applies pressure against the internal surface of the expandable wall resulting in expansion of the wall against the bone thereby augmenting the fixation of the suture anchor in the bone. The pressure applied by the swelling of the swellable copolymer also promotes bone ingrowth into the perforated wall and can be used to increase bone density in the vicinity of the implant. To enhance bone ingrowth, the device of FIG. 4 can be employed in the internal cavity to provide pores in the swellable copolymer for bone ingrowth. Alternatively, a solid swellable copolymer can be employed in the internal cavity.

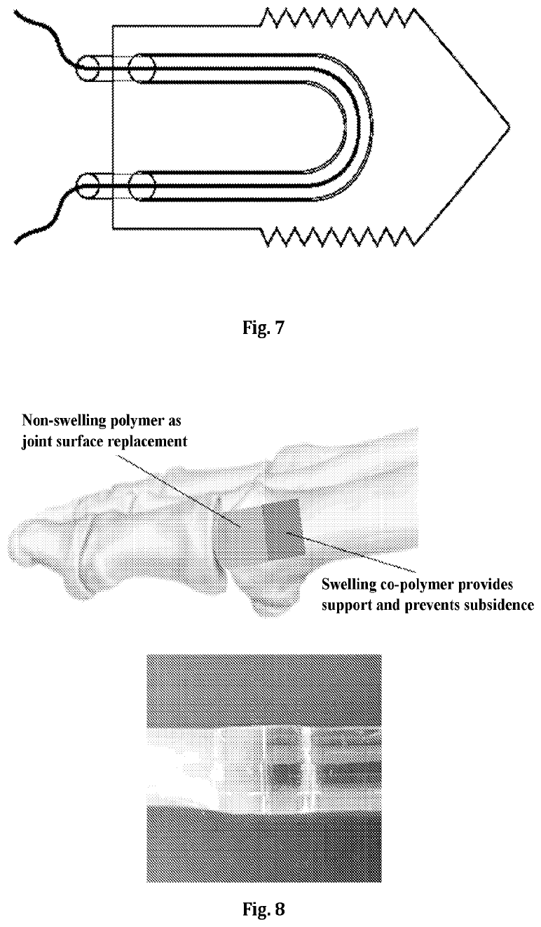

In embodiment 3 of the swelling suture anchor shown in FIG. 7, the entire suture anchor is made of the swelling copolymer and is a combination of solid and porous swelling copolymer. In this case, the suture anchor consists of a solid core of the swelling copolymer surrounded by a porous outer layer of the swelling copolymer. A suture is fixed in the anchor during the copolymerization process either completely exposed or, alternatively, threaded through a thin tube that is located in the copolymer during the copolymerization process in order to shield the suture. Once located in the tube, the suture can slide in the tube or be fixed in the tube or anchor during the polymerization step. The suture of embodiment 3 can be either knotted or knotless. Threads are provided on at least a portion of the outer surface of the suture anchor as shown for initial fixation of the suture anchor to the bone. A sharp tip is provided to facilitate insertion of the anchor into the bone.

The present invention also relates to the methods of making the suture anchors described above, as well as to methods of using the suture anchors for treatment of subjects.

### Osteochondral Defect Repair Systems

An embodiment of the osteochondral defect repair system consists of two parts. The first part is referred to as “artificial cartilage” and consists of either a polyvinyl alcohol (PVA) cryogel or a chemically crosslinked hydrogel. The first part is designed to protrude from the joint surface and provide a smooth surface for articulation with the natural cartilage of the opposing bone. This first part is connected either directly or through an interlock, or other means known in the art to a second part formed from porous swelling copolymer. For example, the swelling part can be provided with a recess into which the first part can be inserted to make the connection.

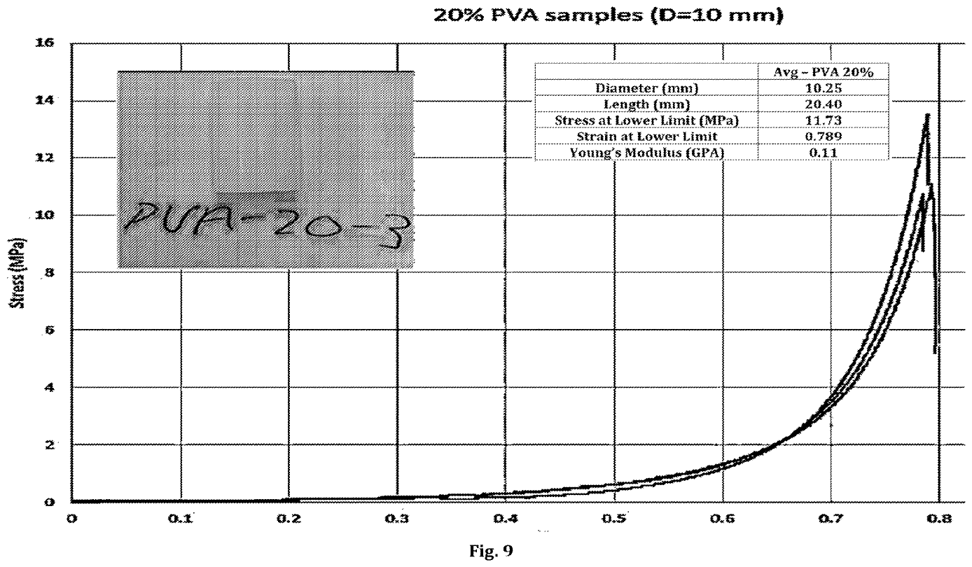

A composite osteochondral defect repair implant for treatment or repair of indications such as first metatarsophalangeal joint osteoarthritis or a talar dome osteochondral defect is shown in FIG. 8. The functional part of the implant that articulates at the joint can be an artificial component such as PVA cryogel, a chemically crosslinked hydrogel or a natural graft (allograft or autograft) such as a cartilage/bone combination from healthy tissue of the patient or from a donor cadaver. The deeper, supporting second part is the porous swelling copolymer that provides the bone fixation and prevents migration of the implant into the bone. For example, the second part of the device can include a small plug of the combination of the solid and porous swelling material that is inserted into the bone to accomplish fixation. The embodiment of FIG. 8 is specifically designed for repair of an osteochondral defect at the metacarpal phalangeal (MTP) joint of the first ray of the foot. This osteochondral repair system was produced and preliminary tests were conducted on the PVA first part to identify the optimal composition for this application. The results are shown in FIG. 9.

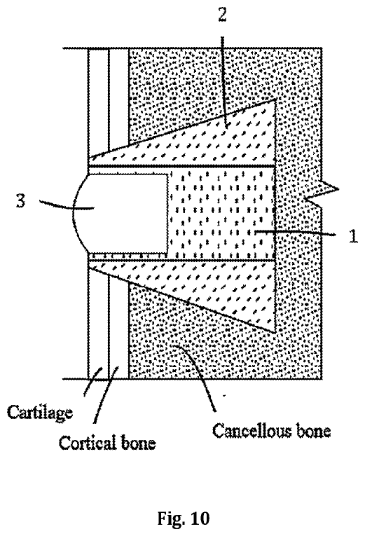

Another embodiment designed for osteochondral defect repair is shown in FIG. 10. In this embodiment, the “artificial cartilage” is embedded inside the porous swelling copolymer thereby providing secure fixation against both proximal migration and movement in the radial direction.

In this second embodiment of the osteochondral defect repair system, the swelling copolymer has a cavity into which the “artificial cartilage” is inserted. The provision of a swelling copolymer with a depth-dependent porosity can be carried out during the molding process or potentially during the printing process

The present invention also relates to methods of making the osteochondral defect repair systems described above, as well as methods of using the osteochondral defect repair systems for treatment of subjects.

Many characteristics of the swelling co-polymer have been extensively investigated and published. These characteristics include:
1. Improved fixation demonstrated by pull out strength experiments,
2. Promotion of bone ingrowth and biocompatibility verified by a series of animal experiments conducted on rabbits, and
3. Stability of the co-polymer by ascertaining that it maintains its integrity and does not deteriorate over a long time period.

Consistent with Wolff's law, the radial stresses produced in the bone by the swelling anchor was shown to promote bone remodeling, resulting in increased bone density in the vicinity of the anchor and a potential for osteointegration [49, 50]. Therefore, the long-term fixation strength of the swelling anchors is expected to increase as compared to the long-term fixation strength of conventional anchors in which bone resorption may occur due to stress-shielding.

In previous studies [47-52] the swelling anchors were solid, not allowing for osteointegration through migration of bone into the anchor. To allow for the long-term benefits of osteointegration, the swelling anchors must possess a porosity to allow bone ingrowth. Consequently, the goal of this study was to develop swelling bone anchors with adequate porosity to promote osteointegration. However, this porosity must not compromise either the swelling properties or the mechanical properties of the anchor. Therefore, the relationship between porosity, swelling, and mechanical properties was an important design criterion for achieving the goals of this study.

This study characterizes the swelling and mechanical properties of three sets of samples made of fully solid, fully porous, and partially porous swelling co-polymeric materials. The goals are that the partially porous swelling co-polymeric sample with a solid core and a porous outer layer should maintain adequate mechanical and swelling properties while the outer layer provides a porous media that may promote mechanically induced osteointegration.

### EXAMPLES

### Specimen Preparation

The process of producing the swelling copolymeric material was described in detail in earlier publications [47, 48, 50]. Therefore, only a summary is provided here. The two crosslinked monomers were methyl methacrylate (MMA), a hydrophobic monomer, and acrylic acid (AA), a hydrophilic monomer. Cross-linking between these two monomers was achieved using diethylene glycol di-methacrylate (DEGDMA). A thermal initiator was added to the mixture (2,2′-azobis 2-methylpropionitrile), and the temperature was slowly raised to 150° C. to achieve full co-polymerization.

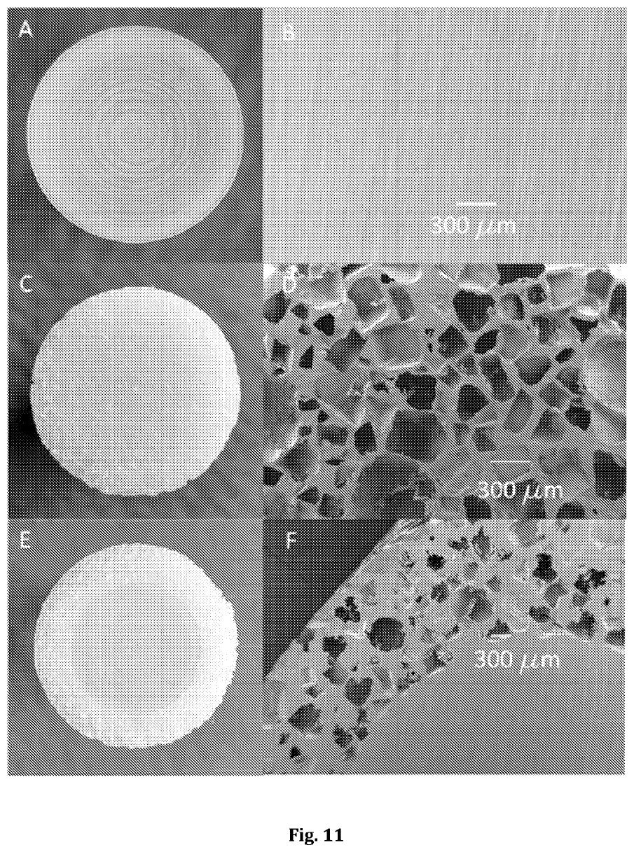

Based on our previous studies [40-43], the optimal mixture that achieves adequate controlled swelling in the range of 4-6 percent by volume for effective fixation to bone, while maintaining adequate mechanical properties was 80% by volume MMA, with 20% by volume AA. To that composition, 5% by volume DEGDMA and 0.3% by weight AIBN were added. Prior to testing, the solid co-polymers were machined into cylindrical samples with an 8 mm diameter and a length of 17 mm (FIG. 11, parts 11A and 11B).

Porous swelling co-polymer samples were produced as follows. NaCl crystals with an average diameter of 300 μm were added to the co-polymer mixture before polymerization. NaCl crystals do not dissolve in the resin and after removing them at the end of the polymerization process through desalination in distilled water, they leave behind a porous, swelling copolymeric structure (FIG. 2) with an average pore size suitable for osteointegration [54, 55]. Once the polymerization and desalination processes were complete, the samples were machined into cylindrical samples with an 8 mm diameter and a length of 17 mm (FIG. 11, parts 11C and 11D).

Preliminary testing of free swelling and compression tests conducted on the porous samples suggested that these samples are mechanically weak, and they further weaken following swelling, possibly rendering them unusable as bone anchors. To address this potential problem a third class of samples were prepared using a procedure that produced samples with a solid swelling core and an outer layer of porous swelling co-polymer that provided the structure for osteointegration.

The process of creating these samples was as follows. First, solid swelling samples were produced as described earlier. These samples were then machined into a cylinder of 5 mm diameter and 17 mm length. These solid samples were then centered carefully into cylindrical tubes with the resin containing the NaCl crystals and a second polymerization process was carried out. Following this second polymerization process, the samples were desalinated to remove the NaCl crystals and machined to achieve an outer diameter of 8 mm. This two-stage polymerization produced samples with a solid 5 mm swelling core and a porous 1.5 mm outer swelling porous layer (FIG. 11D and FIG. 11E). These samples were referred to as hybrid samples. The rationale for these samples was that they would provide adequate mechanical strength while also providing a porous outer layer to promote effective osteointegration.

### Swelling and Mechanical Characterization

The free swelling characteristics of five samples from each of the three sample categories, solid, porous, and hybrid were determined. In actual application of the swelling co-polymer as a bone anchor, the anchor is constrained by the surrounding bone. Therefore, the actual degree of swelling achieved by the material in the implanted condition depends not only on its capacity to swell in the unconstrained (free) condition, but also on factors such as the clearance between the implant and bone, and the magnitude of the pressure at the bone-implant interface.

Free swelling was achieved by immersing each sample in 0.9% saline solution for a period of 60 days while maintaining a constant temperature of 37° C., approximating in vivo conditions. Prior to the beginning of the free swelling test, the diameter and length of the dry unswollen sample was measured with a micrometer (accuracy=0.001 mm), and its weight was measured with an analytical scale (accuracy=0.1 milligram). During the 60 days of swelling, at regular, pre-defined intervals (one day, one week, two weeks, four weeks, six weeks, eight weeks) the sample was removed from the saline solution, wiped dry, and its physical properties were measured before returning the sample to the saline solution for continuation of the free swelling test.

An important feature that enables the swelling co-polymer to act as an effective bone anchor, is its ability to maintain adequate mechanical properties after swelling. Therefore, simple compression tests were conducted on five to 10 samples from each of the three categories, solid, porous, and hybrid. The tests were conducted on both dry samples and on fully swollen samples obtained after 60 days of immersion in 0.9% saline solution. Prior to testing, for each ‘dry’ sample, the diameter, length, weight, and density of the sample were recorded. For the ‘swollen’ samples, these measurements were performed both at the beginning and at the end of the swelling period. The mechanical tests were conducted at room temperature with a mechanical testing system (Mark-10 ESM 1500) using a slow crosshead speed of 1 mm/min while data on force (N) and displacement (mm) were recorded. Samples were compressed until failure was observed or until the strain exceeded 0.3, at which point, the resultant large deformation of the sample was considered to render it non-functional as a bone anchor.

### Data Processing

Swelling characterization: The surface area of the samples (2πrl—where r is the sample diameter and l is its length) was used as a parameter to characterize swelling. The choice of this parameter was based on the fact that during actual applications, fixation of the swelling sample to bone was based on the entire surface of the sample that is in contact with the bone. Repeated Measure Analysis of Variance (MANOVA) was used to address specific questions including:
1. Were there significant differences between the three groups of samples (solid, porous, and hybrid) in their swelling characteristics?
2. Were there significant differences between short-term, mid-term, and long-term swelling properties?

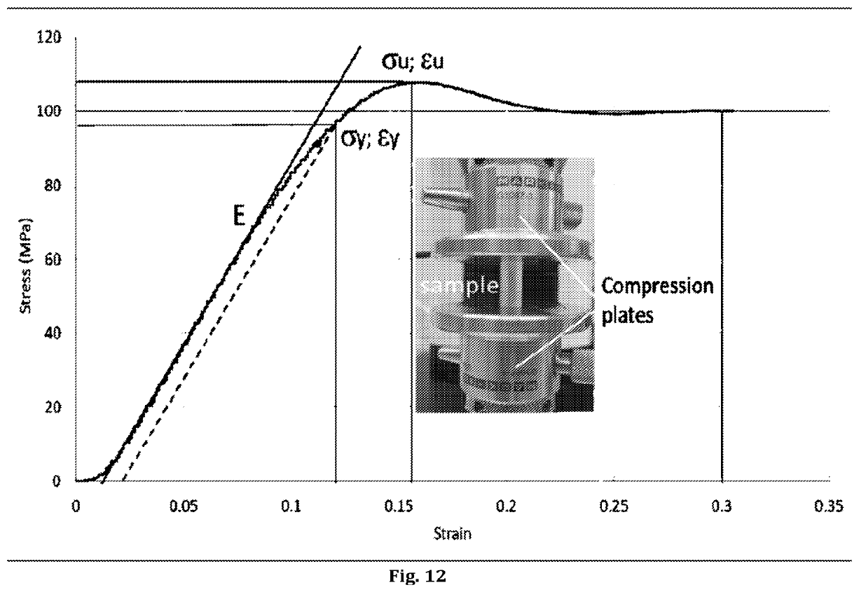

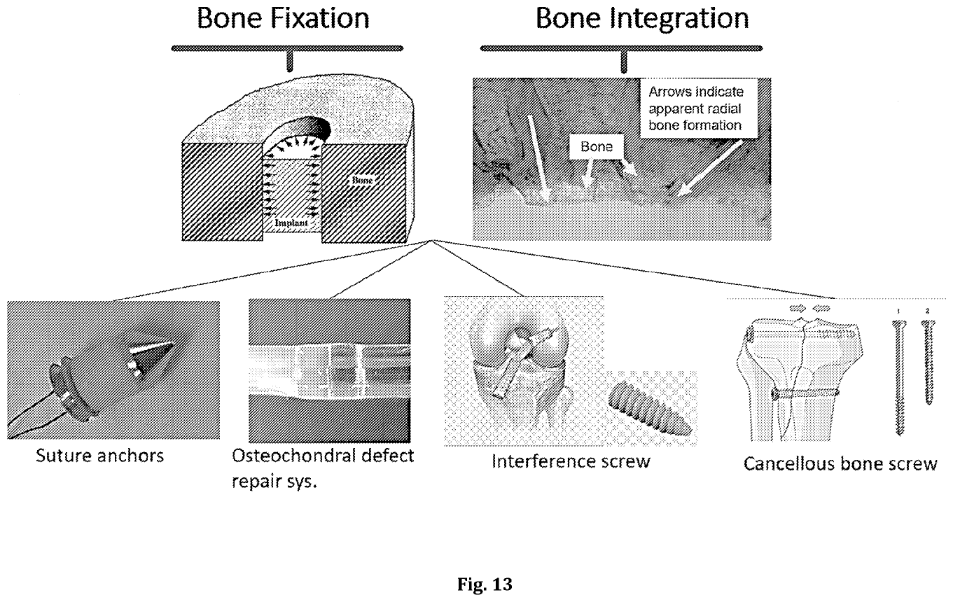

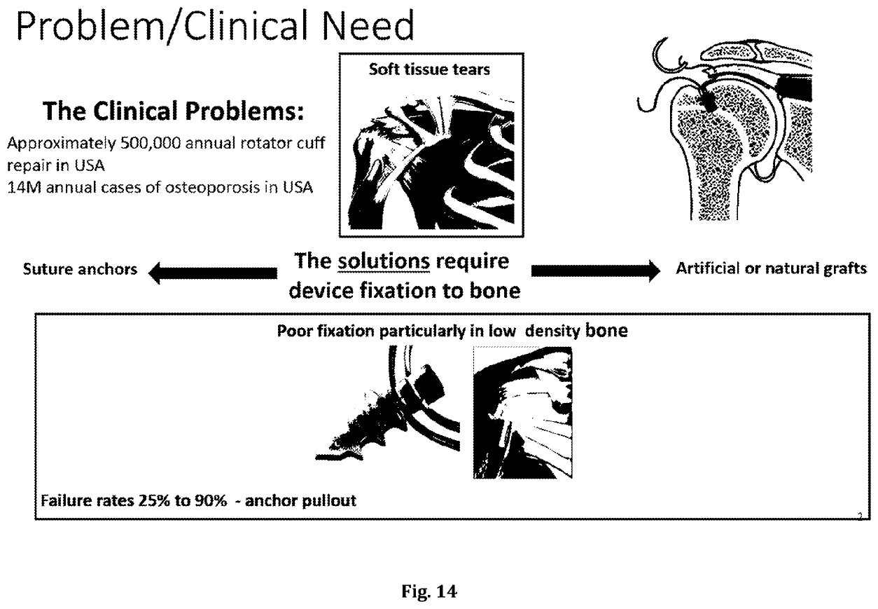

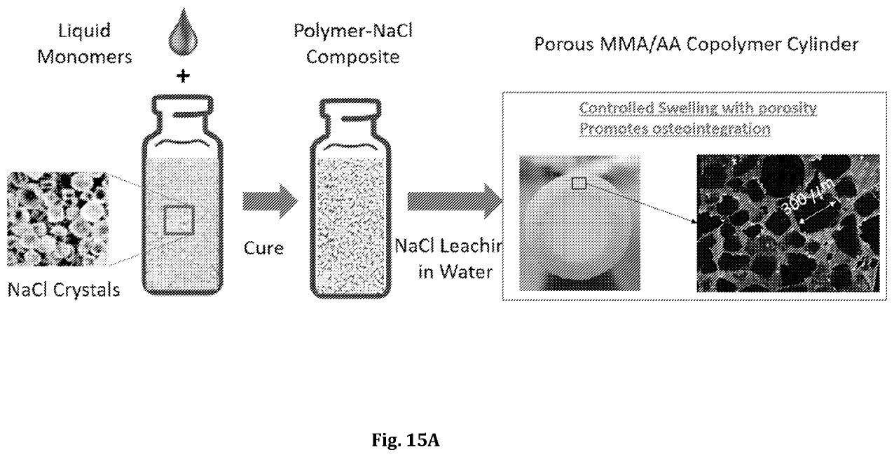

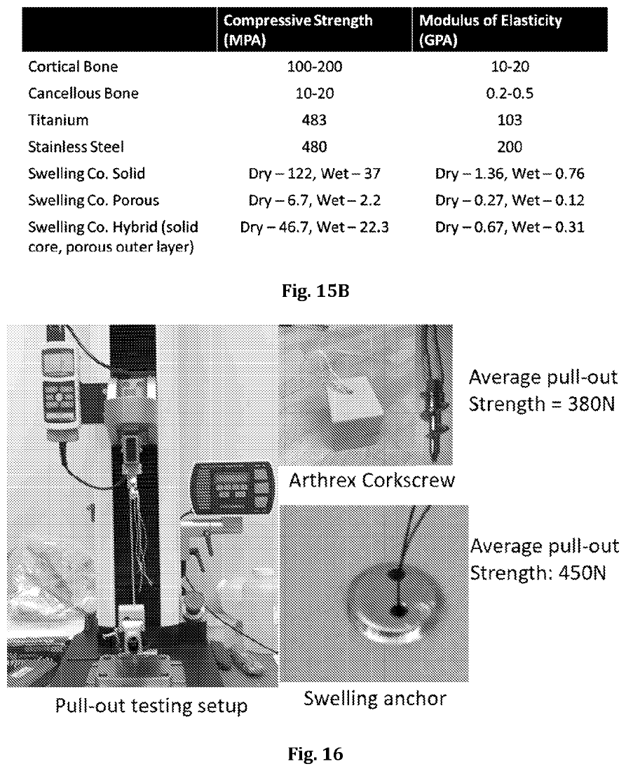

Mechanical characterization: From the force vs. displacement data obtained during the compression mechanical testing (FIG. 12), a number of mechanical properties were extracted including modulus of elasticity (slope of the stress-strain curve in the initial, linear region); ultimate stress and ultimate strain (local maximum of the stress-strain curve after the initial linear region); yield stress and yield strain identified as the point of the stress-strain curve offset by 5% of the ultimate strain; and the toughness of the material defined as the area under the stress-strain curve to a strain value of 0.3 (FIG. 12). These properties were obtained for each sample type both under the ‘dry’, non-swelling condition, and under ‘wet’ conditions following sixty days of swelling in 0.9% saline solution. Analysis of Variance (ANOVA) was used to address the following questions:
1) Were there significant differences in mechanical properties between ‘dry’ and ‘wet’ samples?
2) Were there significant differences in mechanical properties between the three sample groups?

### Results

The results of the free swelling characterization (Table 1) showed significant differences in swelling behavior between the three sample types (solid, hybrid, and porous). All sample types (solid, hybrid, and porous) showed a quick initial swelling that slowed as time progresses and reaches a close to maximum swelling at 60 days. The solid samples swelled significantly less than the hybrid and the porous samples both in the short term of one day and the long term of one month. Comparison of the swelling of the porous samples to the hybrid samples showed that in the short terms of one day and one week the porous sample swelled significantly more than the hybrid samples. However, in the longer-terms of two weeks and one month, the differences in swelling between these sample types was not statistically significant although the porous samples swelled slightly more than the hybrid ones. See Table 1.

In terms of the swelling rates, the porous samples swelled quickly during the first day reaching 74% of their maximum free swelling. The rate of swelling then slowed in comparison to the other two sample types and by one week had further increased the surface area by an average of only 6% reaching 80% of maximum. The solid samples swelled the least in one day reaching only 21% of maximum but continued to swell, reaching 64% of maximum within one week. The hybrid sample reached 39% of maximum in one day and 63% of maximum in one week.

TABLE 1

| | | | | 95% Confidence Interval | |
|---|---|---|---|---|---|
| Sample | Swelling | | | Lower | Upper |
| type | Time | Mean | Std. Error | bound | Bound |
| Hybrid | One day | 2.03 | 0.26 | 1.48 | 2.57 |
| | One week | 3.28 | 0.32 | 2.61 | 3.95 |
| | Two weeks | 4.53 | 0.28 | 3.95 | 5.12 |
| | One month | 5.23 | 0.27 | 4.67 | 5.80 |
| Porous | One day | 4.39 | 0.26 | 3.84 | 4.93 |
| | One week | 4.74 | 0.32 | 4.07 | 5.41 |
| | Two weeks | 5.37 | 0.28 | 4.78 | 5.95 |
| | One month | 5.95 | 0.27 | 5.38 | 6.51 |
| Solid | One day | 0.91 | 0.18 | 0.53 | 1.30 |
| | One week | 2.73 | 0.22 | 2.26 | 3.20 |
| | Two weeks | 3.06 | 0.20 | 2.64 | 3.47 |
| | One month | 4.25 | 0.19 | 3.85 | 4.65 |

The results of the mechanical characterization in Table 2 below show that swelling of the samples in all three groups (solid, hybrid, and porous) resulted in significant reductions in their yield stress, ultimate stress, modulus of elasticity, and toughness. Yield strain decreased significantly with swelling only in the solid group, but ultimate strain decreased significantly with swelling in all three groups. Comparing the sample groups showed that both under ‘dry’ and ‘wet’ (swelled) conditions the yield stress, ultimate stress, modulus of elasticity, and toughness were significantly different between the three groups with the highest values in the solid group, followed by the hybrid group and lowest in the porous group. Both yield strain and ultimate strain were not significantly different when comparing the solid and the hybrid group. However, both strain values were significantly lower in the porous group when compared both to the solid and the hybrid group.

TABLE 2

| | Solid | | Porous | | Hybrid | |
|---|---|---|---|---|---|---|
| | Dry | Wet | Dry | Wet | Dry | Wet |
| Yield Stress [MPa] | 104.60 | 40.15 | 7.54 | 3.32 | 50.30 | 24.78 |
| St. Dev. | 9.62 | 2.10 | 0.30 | 0.65 | 2.24 | 1.96 |
| Yield Strain | 0.12 | 0.07 | 0.04 | 0.03 | 0.11 | 0.11 |
| [mm/mm] | | | | | | |
| St. Dev. | 0.01 | 0.003 | 0.004 | 0.01 | 0.01 | 0.02 |
| Ultimate Stress | 119.85 | 43.89 | 9.42 | 3.85 | 56.79 | 26.60 |
| [MPa] | | | | | | |
| St. Dev. | 11.74 | 2.50 | 0.43 | 0.60 | 2.70 | 0.98 |
| Ultimate Strain | 0.17 | 0.09 | 0.07 | 0.04 | 0.15 | 0.12 |
| [mm/mm] | | | | | | |
| St. Dev. | 0.01 | 0.004 | 0.002 | 0.01 | 0.004 | 0.01 |
| Young's Modulus | 0.97 | 0.64 | 0.27 | 0.12 | 0.55 | 0.26 |
| [GPa] | | | | | | |
| St. Dev. | 0.01 | 0.03 | 0.02 | 0.01 | 0.06 | 0.03 |
| Toughness [J/m³] | 2.65E+07 | 1.07E+07 | 2.73E+06 | 1.11E+06 | 1.35E+07 | 6.26E+06 |
| St. Dev. | 2.04E+06 | 7.35E+05 | 1.61E+05 | 1.53E+05 | 7.01E+05 | 2.16E+05 |

### Discussion

The swelling co-polymer utilized in this present study produced radial stresses at the implant-bone interface through material swelling. The magnitude of these stresses can be controlled by the amount of swelling of the co-polymer. The radial stresses in the bone produced the desired stimulus of mechano-transduction to induce bone remodeling. In previous studies conducted with solid cylindrical swelling implants in a rabbit model, this swelling process enhanced bone remodeling. Histological studies revealed intimate bone deposition at the bone-implant interface with no fibrous tissue present [50]. However, in those studies the benefits of swelling implant porosity were not investigated. The challenge and goal of the present study was to produce a swelling implant with adequate porosity. Such an implant will have the advantage of promoting bone remodeling through swelling and induced stresses in the bone and, at the same time, providing porosity for effective osteointegration. However, the mechanical properties of such implants must not be compromised to an extent rendering them useless for specific applications.

Controlled porosity has been achieved in swelling co-polymers through a simple and cost-effective process. Fully porous bone anchors swelled and their mechanical properties were significantly reduced following immersion in saline solution rendering these properties significantly worse than those of cortical or cancellous bone.

TABLE 3

| | Compressive Strength | Modulus of Elasticity |
|---|---|---|
| | (MPa) | (GPa) |
| Cortical Bone | 131-224 | 17-20 |
| Cancellous Bone | 0.15-100 | 0.02-0.8 |
| Titanium Alloy | 900 | 110-127 |
| Stainless Steel | 500-1000 | 180-205 |
| PEEK | 120-140 | 3.0-4.0 |
| Swelling Co. Solid | 40.15 | 0.64 |
| Swelling Co. Porous | 3.32 | 0.12 |
| Swelling Co. Hybrid | 24.78 | 0.26 |

Table 3 is a comparison of the mechanical properties of the types of swelling co-polymers (solid, porous, and hybrid), both ‘dry’ and ‘wet” (fully swollen) to the properties of cortical and cancellous bone and the properties of artificial materials currently used in orthopedic implants such as titanium, stainless steel, and PEEK. The values for the cortical and cancellous bone and for the artificial materials were obtained from a recent review article. [79].

In order to overcome the mechanical problems of using 100% porous material, the hybrid anchor was developed. The hybrid anchor includes a solid swelling core and an outer layer of a swelling porous structure with effective porosity for osteo-conduction and osteointegration (FIG. 11). The hybrid swelling bone anchors have several important properties. Compared to the fully porous swelling anchors, they maintained mechanical properties following swelling that were close to those of cancellous bone while the fully porous swelling anchors become mechanically too weak to act as effective anchors (Table 3). These bone-matching properties of the hybrid swelling anchors show great promise for providing a more effective bone-implant interface by mitigating stress shielding due to mismatch in mechanical properties. Such a mismatch in mechanical properties is obvious in bone anchors made of titanium or stainless steel (Table 3) with mechanical properties greatly exceeding those of bone thus producing stress-shielding effects in the surrounding bone.

A porous swelling co-polymeric material was made and its swelling and mechanical properties in three different configurations (solid, fully porous, and partially porous (hybrid) were determined. Based on the results, it was concluded that the hybrid, porous swelling material can be configured as a bone anchor with several important potential advantages over conventional commercial bone anchors. These include maintaining closely matched mechanical properties to cancellous bone; providing a fixation and refixation mechanism based on friction rather than shear, thus making it particularly effective in low density bone such as osteoporotic bone; and mechanically inducing radial stresses due to swelling, triggering remodeling and osteointegration.

### Refixation Properties

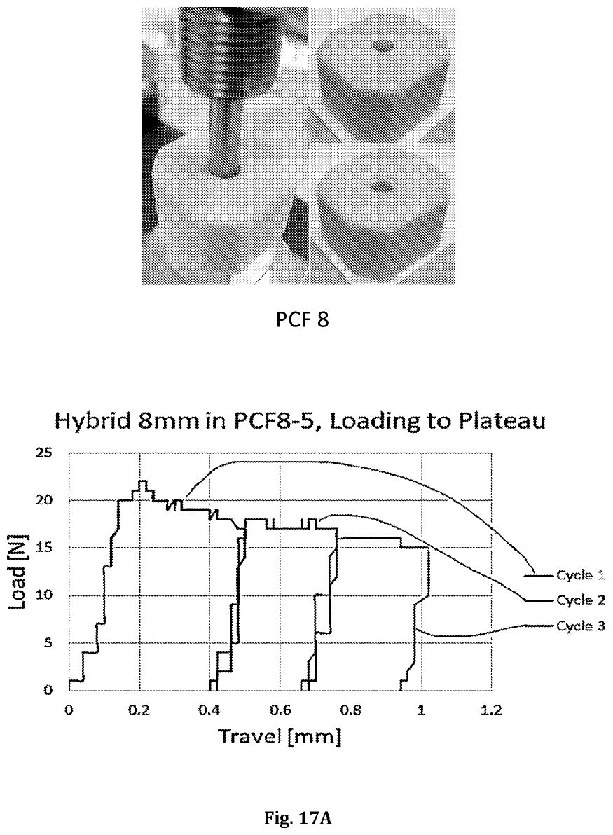

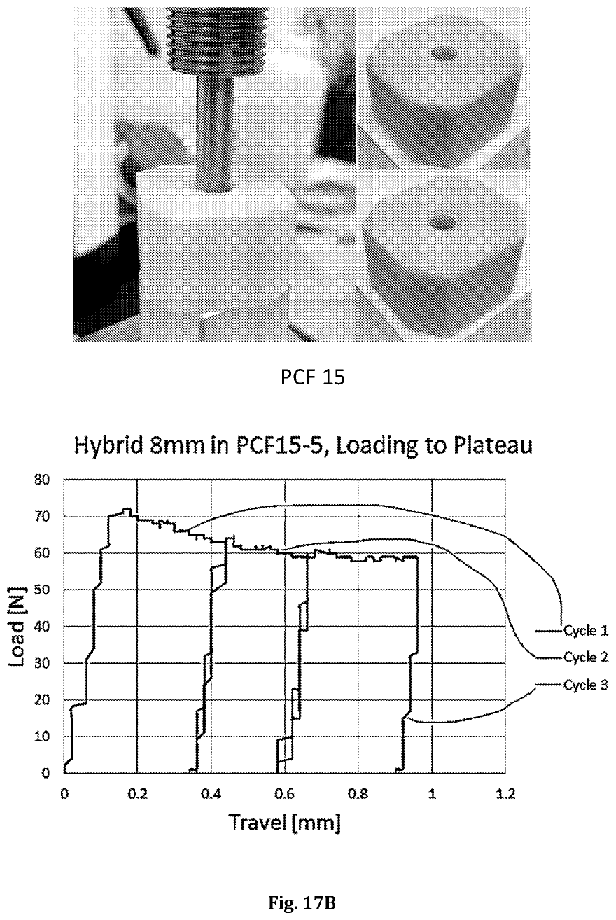

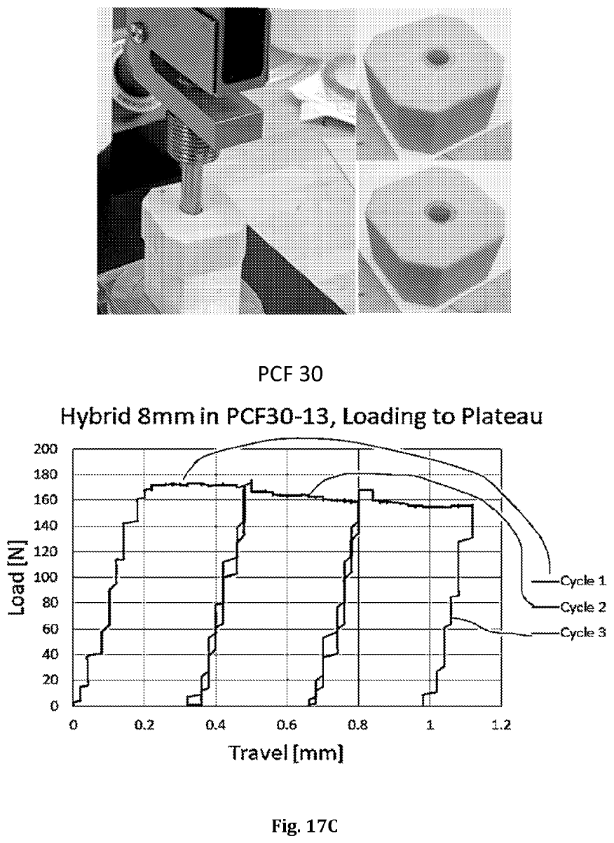

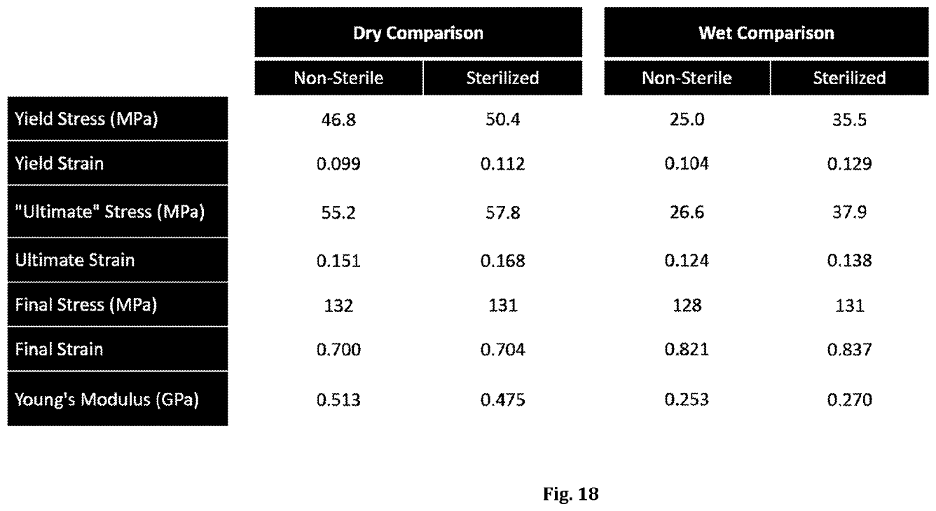

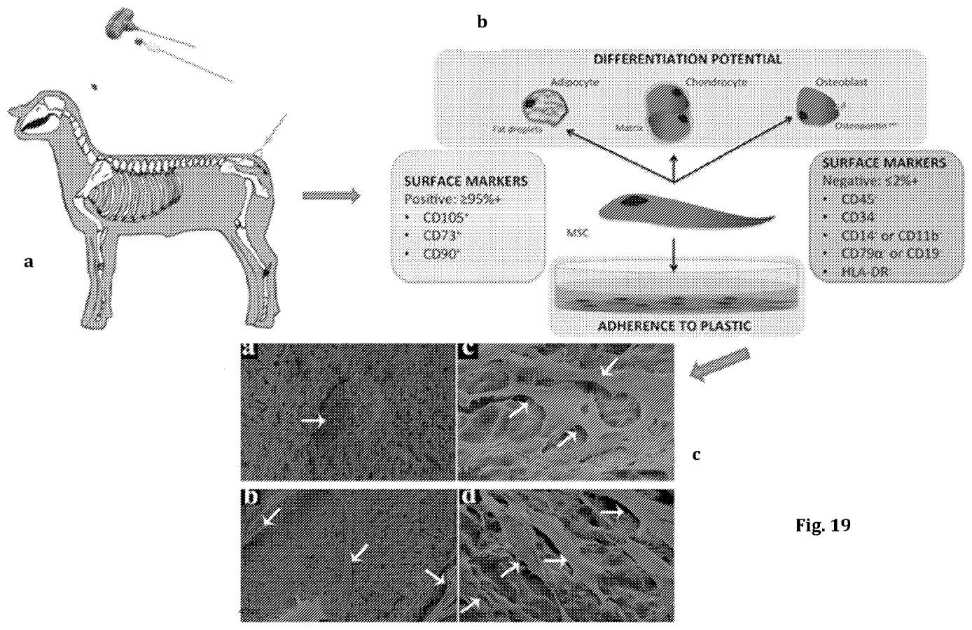

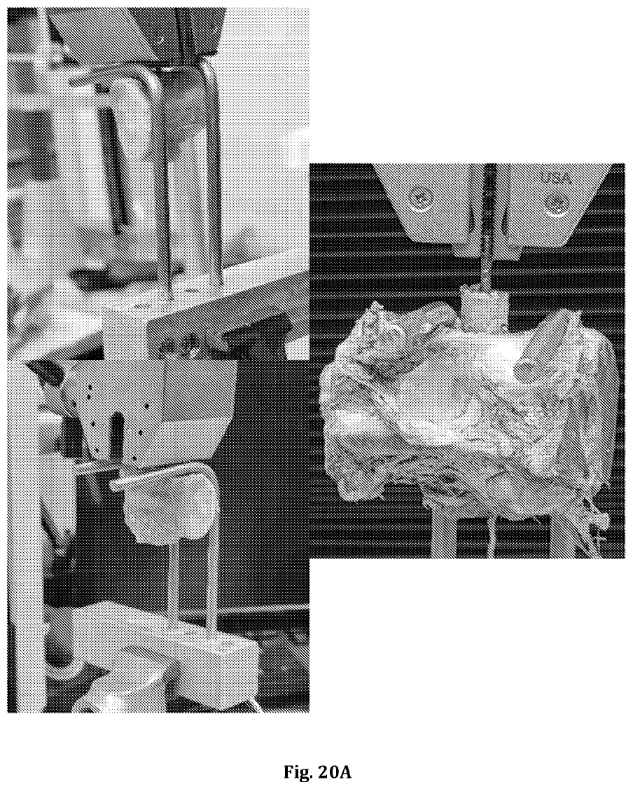

The swelling anchors were tested for refixation properties. The results are shown in FIGS. 17A-17C. The refixation properties of the swelling anchors were tested by increasing a load until the swelling anchor exceeded frictional resistance and moved slightly. This was considered one cycle. The load is then removed for a period of time before carrying out the next cycle. This was repeated for a total of three cycles. As shown in FIGS. 17A-17C, after the load was removed, the resting force receded to the previous level. This indicates that the bone anchor did not lose fixation properties after the load was applied. In contrast, conventional screws lose their fixation strength once a force on the conventional screw exceeds its fixation strength.

### In Vitro Pull-Out Study

An in vitro pull-out study was performed using anchors implanted in the lumbar vertebrae of cadaver sheep.

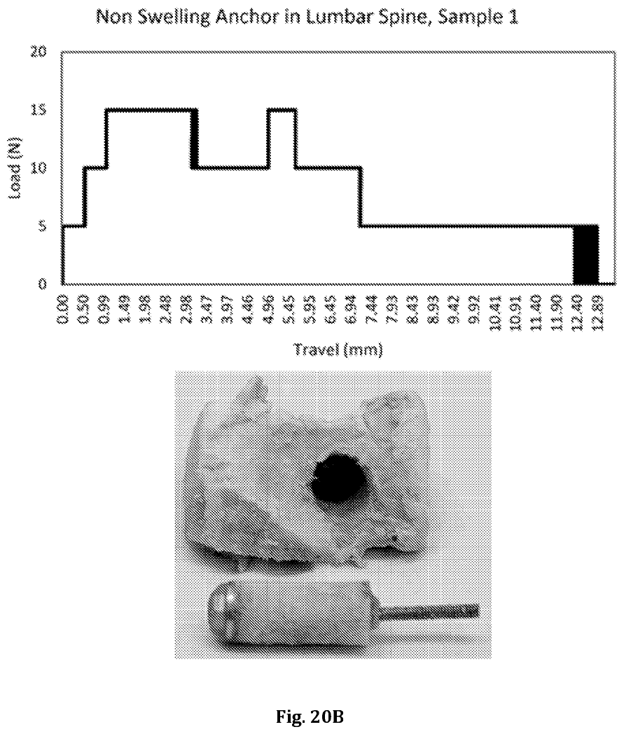

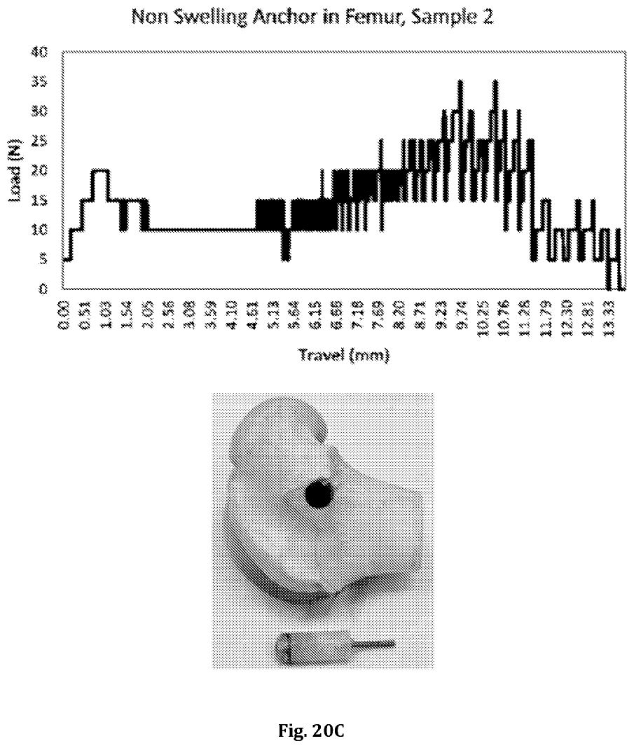

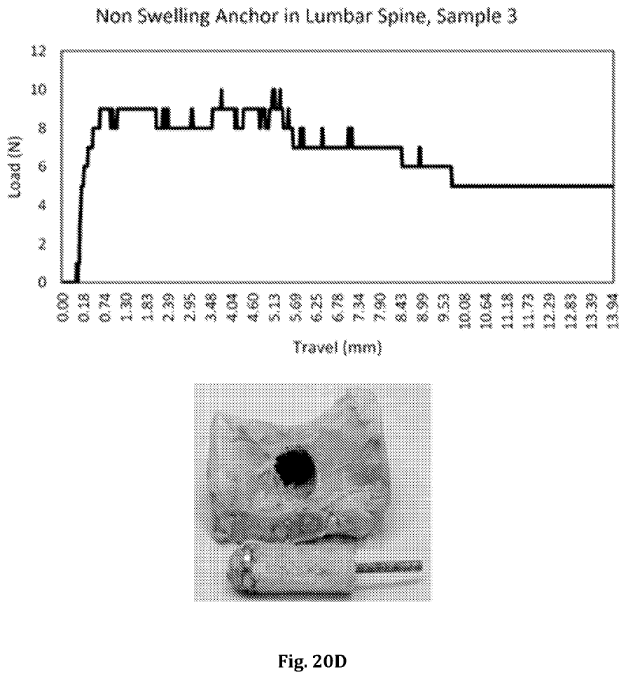

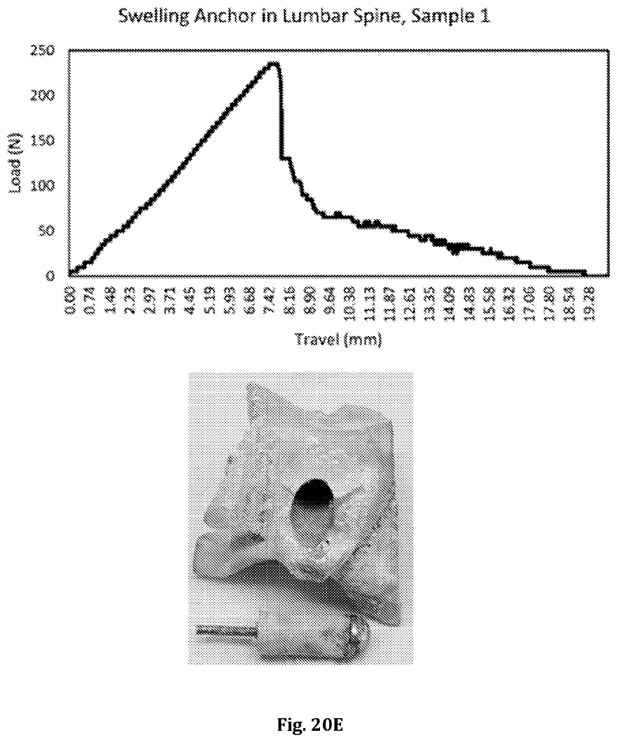

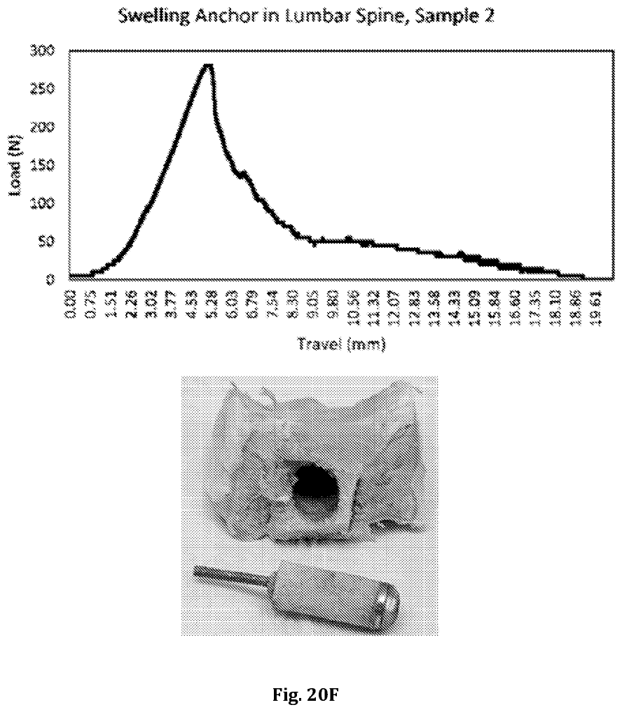

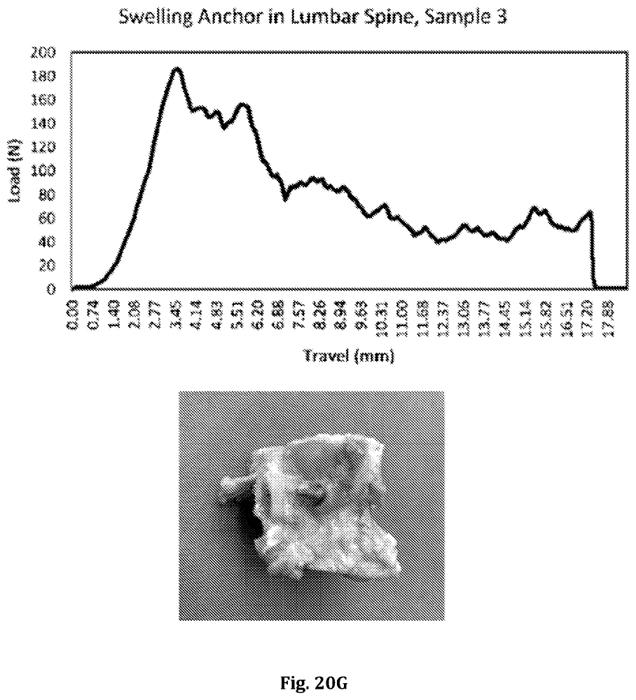

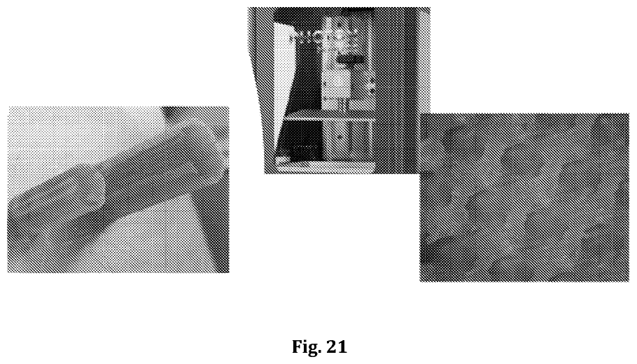

Three conventional non-swelling anchors (See FIGS. 20B-20D) and three swelling anchors according to the present invention (See FIGS. 20E-20G) were separately implanted in different vertebrae and allowed to swell for one week. At the end of the week, the implanted vertebrae were mounted onto a tensile testing machine, as shown in FIG. 20A, and the anchor samples were each pulled out. The tensile testing machine measured the force required for the pull-out until the sample was completely dislodged from the bone.

When comparing the results from FIGS. 20B-20D which employed conventional devices with the results of FIGS. 20E-20G which employed devices of the invention, the swelling anchors according to the present invention required significantly more pull out force than the conventional non-swelling anchors to completely dislodge the anchors from the bone. For example, FIGS. 20E-20G demonstrating the results of the swelling anchors of the present invention required a pull out force in excess of 180 N up to 250 N, whereas FIGS. 20B-20D demonstrating the results of the conventional non-swelling anchors only required a pull out force of less than 35 N to completely dislodge the anchors from the bone. Force was determined using a tensile testing machine.

### REFERENCES

All references cited herein are hereby incorporated by reference in their entirety as if fully set forth herein.
1. Hamid K S, Parekh S G. Clinical Presentation and Management of Hallux Rigidus. Foot Ankle Clin. 2015; 20(3): 391-9. Epub 2015 Sep. 1. doi: 10.1016/j.fcl.2015.04.002. PubMed PMID: 26320554.
2. Kennedy J G, Chow F Y, Dines J, Gardner M, Bohne W H. Outcomes after interposition arthroplasty for treatment of hallux rigidus. Clin Orthop Relat Res. 2006; 445:210-5. Epub 2006 Feb. 10. doi: 10.1097/01.blo.0000201166.82690.23. PubMed PMID: 16467621.
3. Giannini S, Buda R, Grigolo B, Vannini F. Autologous chondrocyte transplantation in osteochondral lesions of the ankle joint. Foot Ankle Int. 2001; 22(6): 513-7. Epub 2001 Jul. 28. doi: 10.1177/107110070102200612. PubMed PMID: 11475462.
4. J. Paul, A. Barg, G. Pagensteert, V. Valderrabano, Imhoff A B. Osteochondral Injuries of the Talus, In book: Sports injuries: Prevention, diagnosis, treatment and rehabilitation. Springer, editor 2015.
5. Abusafieh A, Siegler S, Kalidindi S R. Development of self-anchoring bone implants. I. Processing and material characterization. J Biomed Mater Res. 1997; 38(4): 314-27.
6. Vemuganti A, Siegler S, Abusafieh A, Kalidindi S. Development of self-anchoring bone implants. II. Bone-implant interface characteristics in vitro. J Biomed Mater Res. 1997; 38(4): 328-36. PubMed PMID: 9421753.
7. Nien Y H, Kalidindi S R, Siegler S. Fixation strength of swellable bone anchors in low-density polyurethane foam. J Biomed Mater Res. 2001; 58(2): 137-46. Epub 2001 Mar. 10. doi: 10.1002/1097-4636(2001)58:2<137:AID-JBM1000>3.0.CO; 2-S [pii]. PubMed PMID: 11241332.
8. Gualtieri G M, Siegler S, Hume E L, Kalidindi S R. Biological and mechanical characteristics of the interface between a new swelling anchor and bone. J Orthop Res. 2000; 18(3): 494-9. doi: 10.1002/jor.1100180324. PubMed PMID: 10937639.
9. Colvin A C, Egorova N, Harrison A K, Moskowitz A, Flatow E L. National trends in rotator cuff repair. J Bone Joint Surg Am. 2012; 94(3): 227-33. Epub 2012 Feb. 3. doi: 10.2106/JBJS.J.00739. PubMed PMID: 22298054; PMCID: PMC3262185.
10. Siegler S, inventor; Drexel University, assignee. swelling type copolymeric composite materials with self-fixation characteristics. U.S. Pat. No. 5,824,079. 1998.
11. Khazen, G. E., et al., Fixation of calcaneal avulsion fractures using screws with and without suture anchors: a biomechanical investigation. Foot & ankle international, 2007. 28(11): p. 1183-1186.
12. Kim, K.-S., et al., Suture anchor fixation of comminuted inferior pole patella fracture novel technique: suture bridge anchor fixation technique. Archives of Orthopaedic and Trauma Surgery, 2021. 141(11): p. 1889-1897.
13. Ravenscroft, M. J., et al., Suture anchor fixation of olecranon fractures: a case series. Shoulder & Elbow, 2013. 5(2): p. 116-119. 14. Taljanovic, M. S., et al., Fracture fixation. Radiographics, 2003. 23(6): p. 1569-1590.
15. Cho, B.-K., et al., A prospective outcome and cost-effectiveness comparison between two ligament reattachment techniques using suture anchors for chronic ankle instability. Foot & ankle international, 2015. 36(2): p. 172-179.
16. Ducic, Y., Medial canthal ligament reattachment in skull base surgery and trauma. The Laryngoscope, 2001. 111(4): p. 734-737.
17. Harwin, S. F., Osteolysis of the greater trochanter: a result of bone anchors used for abductor reattachment at total hip arthroplasty. The Journal of arthroplasty, 2006. 21(1): p. 97-101.
18. Maffulli, N., et al., Calcific insertional Achilles tendinopathy: reattachment with bone anchors. The American Journal of Sports Medicine, 2004. 32(1): p. 174-182.
19. Barber, F. A., et al., Arthroscopic Bankart reconstruction with a bioabsorbable anchor. Journal of shoulder and elbow surgery, 2003. 12(6): p. 535-538.
20. Frank, R. and A. Romeo, Arthroscopic soft tissue reconstruction in anterior shoulder instability. Der Orthopäde, 2018. 47(2): p. 121-128.
21. Shall, L. M. and P. W. Cawley, Soft tissue reconstruction in the shoulder: Comparison of suture anchors, absorbable staples, and absorbable tacks. The American Journal of Sports Medicine, 1994. 22(5): p. 715-718.
22. Ozturk, S., et al., Correction of bilateral congenital swan-neck deformity by use of Mitek mini anchor: a new technique. British journal of plastic surgery, 2005. 58(6): p. 822-825.
23. Warren, B., et al., Use of Ultrasonically Interdigitated Anchors in Haglund's Deformity Correction Surgery. Foot & Ankle Specialist, 2021. 14(2): p. 148-152.
24. Bazaz, R. and R. D. Ferkel, Treatment of osteochondral lesions of the talus with autologous chondrocyte implantation. Techniques in Foot & Ankle Surgery, 2004. 3(1): p. 45-52.
25. Maugans, C. J., et al., Tibial tubercle osteotomy: A biomechanical comparison of two techniques. The Knee, 2017. 24(2): p. 264-270.
26. Tuncer, C., et al., Osteotomy assisted maxillary posterior impaction with miniplate anchorage. The Angle Orthodontist, 2008. 78(4): p. 737-744.
27. Tsui, W., H. Chua, and L. Cheung, Bone anchor systems for orthodontic application: a systematic review. International journal of oral and maxillofacial surgery, 2012. 41(11): p. 1427-1438.
28. Visscher, L. E., et al., The history of suture anchors in orthopaedic surgery. Clinical Biomechanics, 2019. 61: p. 70-78.
29. Chapman, J., et al., Factors affecting the pullout strength of cancellous bone screws. Journal of biomechanical engineering, 1996. 118(3): p. 391-398.
30. Fletcher, J. W., et al., Surgical performance when inserting non-locking screws: a systematic review. EFORT Open Reviews, 2020. 5(1): p. 26-36.
31. Aworinde, A. K., et al., Mechanical strength and biocompatibility properties of materials for bone internal Fixation: a brief overview. 2018.
32. Li, J., et al., Materials evolution of bone plates for internal fixation of bone fractures: A review. Journal of Materials Science & Technology, 2020. 36: p. 190-208.
33. Chamay, A. and P. Tschantz, Mechanical influences in bone remodeling. Experimental research on Wolff's law. Journal of biomechanics, 1972. 5(2): p. 173-180.
34. Lanyon, L. and S. Bourn, The influence of mechanical function on the development and remodeling of the tibia. An experimental study in sheep. JBJS, 1979. 61(2): p. 263-273.
35. Dhawan, A., et al., Complications of bioabsorbable suture anchors in the shoulder. The American journal of sports medicine, 2012. 40(6): p. 1424-1430.
36. Kurtz, S. M. and J. N. Devine, PEEK biomaterials in trauma, orthopedic, and spinal implants. Biomaterials, 2007. 28(32): p. 4845-4869.
37. Barber, F. A., et al., The relationship of suture anchor failure and bone density to proximal humerus location: a cadaveric study. Arthroscopy: The Journal of Arthroscopic & Related Surgery, 1997. 13(3): p. 340-345.
38. Barber, F. A., M. A. Herbert, and J. N. Click, The ultimate strength of suture anchors. Arthroscopy: The Journal of Arthroscopic & Related Surgery, 1995. 11(1): p. 21-28.
39. Craft, D. V., et al., Fixation strength of rotator cuff repair swith suture anchors and the transosseous suture technique. Journal of shoulder and elbow surgery, 1996. 5(1): p. 32-40.
40. Djurasovic, M., et al., Revision rotator cuff repair: factors influencing results. JBJS, 2001. 83(12): p. 1849-1855.
41. Gerber, C., B. Fuchs, and J. Hodler, The results of repair of massive tears of the rotator cuff. JBJS, 2000. 82(4): p. 505.
42. Hecker, A. T., et al., Pull-out strength of suture anchors for rotator cuff and Bankart lesion repairs. The American Journal of Sports Medicine, 1993. 21(6): p. 874-879.
43. St Pierre, P., et al., Tendon-healing to cortical bone compared with healing to a cancellous trough. A biomechanical and histological evaluation in goats. The Journal of bone and joint surgery. American volume, 1995. 77(12): p. 1858-1866.
44. Jenkins, D. P. and T. H. Cochran, 15 Osteoporosis: The Dramatic Effect of Disuse of an Extremity. Clinical Orthopaedics and Related Research®, 1969. 64: p. 128-134.
45. Joffre, T., et al., Trabecular deformations during screw pull-out: a micro-CT study of lapine bone. Biomechanics and Modeling in Mechanobiology, 2017. 16(4): p. 1349-1359.
46. Tjellström, A., et al., Direct bone anchorage of external hearing aids. Journal of biomedical engineering, 1983. 5(1): p. 59-63.
47. Abusafieh, A., R. Gobran, and S. R. Kalidindi, Synthesis and characterization of a poly (methyl methacrylate-acrylic acid) copolymer for bioimplant applications. Journal of applied polymer science, 1997. 63(1): p. 75-87.
48. Abusafieh, A., S. Siegler, and S. R. Kalidindi, Development of self-anchoring bone implants. I. Processing and material characterization. Journal of biomedical materials research, 1997. 38(4): p. 314-327.
49. Greenberg, A., et al., Stimulation of bone formation by a swelling endosseous implant. Journal of Biomedical Materials Research, 1978. 12(6): p. 929-933.
50. Gualtieri, G. M., et al., Biological and mechanical characteristics of the interface between a new swelling anchor and bone. Journal of Orthopaedic Research, 2000. 18(3): p. 494-499.
51. Kalidindi, S. R. and P. Ahmad, A Numerical Investigation of the Mechanics of Swelling-Type Intramedullary Hip Implants. Journal of Biomechanical Engineering, 1997. 119(3): p. 241-247.
52. Nien, Y. H., S. R. Kalidindi, and S. Siegler, Fixation strength of swellable bone anchors in low-density polyurethane foam. J Biomed Mater Res, 2001. 58(2): p. 137-46
53. Vemuganti, A., et al., Development of self-anchoring bone implants. II. Bone-implant interface characteristics in vitro. J Biomed Mater Res, 1997. 38(4): p. 328-36.
54. Boyan, B. D., et al., Role of material surfaces in regulating bone and cartilage cell response. Biomaterials, 1996. 17(2): p. 137-146.
55. Freyman, T., I. Yannas, and L. Gibson, Cellular materials as porous scaffolds for tissue engineering. Progress in Materials science, 2001. 46(3-4): p. 273-282.
56. Coelho, P. G., et al., Osseointegration: hierarchical designing encompassing the macrometer, micrometer, and nanometer length scales. Dental Materials, 2015. 31(1): p. 37-52.
57. Li, J., et al., Development of a porous 3D graphene-PDMS scaffold for improved osseointegration. Colloids and Surfaces B: Biointerfaces, 2017. 159: p. 386-393.
58. Souza, J. C., et al., Nano-scale modification of titanium implant surfaces to enhance osseointegration. Acta biomaterialia, 2019. 94: p. 112-131.
59. Hsieh, Y.-Y., et al., Pull-Out Capability of a 3D Printed Threadless Suture Anchor with Rectangular Cross-Section: A Biomechanical Study. Applied Sciences, 2021. 11(24): p. 12128.
60. Tai, C.-C., et al., Biocompatibility and biological performance evaluation of additive manufactured bioabsorbable iron-based porous suture anchor in a rabbit model. International journal of molecular sciences, 2021. 22(14): p. 7368.
61. Takemoto, M., et al., Mechanical properties and osteoconductivity of porous bioactive titanium. Biomaterials, 2005. 26(30): p. 6014-6023.
62. Vasconcellos, L. M. R. d., et al., Novel production method of porous surface Ti samples for biomedical application. Journal of Materials Science: Materials in Medicine, 2012. 23(2): p. 357-364.
63. Apostu, D., et al., Current methods of preventing aseptic loosening and improving osseointegration of titanium implants in cementless total hip arthroplasty: a review. J Int Med Res, 2018. 46(6): p. 2104-2119.
64. Apostu, D., et al., Systemic drugs that influence titanium implant osseointegration. Drug Metab Rev, 2017. 49(1): p. 92-104.
65. Beck, F., et al., Immobilization of Denosumab on Titanium Affects Osteoclastogenesis of Human Peripheral Blood Monocytes. Int J Mol Sci, 2019. 20(5).
66. Lai, M., X. Yan, and Z. Jin, The response of bone cells to titanium surfaces modified by simvastatin-loaded multilayered films. J Biomater Sci Polym Ed, 2018. 29(15): p. 1895-1908.
67. Tang, J., et al., Enhancement of local bone formation on titanium implants in osteoporotic rats by biomimetic multilayered structures containing parathyroid hormone (PTH)-related protein. Biomed Mater, 2020. 15(4): p. 045011.
68. Jurczak P, W. J., Rodziewicz-Motowidło S, Lach S., Proteins, peptides and peptidomimetics as active agents in implant surface functionalization. Adv Colloid Interface Sci., 2020. 276(102083).
69. Zavan, B., et al., Release of VEGF from Dental Implant Improves Osteogenetic Process: Preliminary In Vitro Tests. Materials (Basel), 2017. 10(9).
70. Zhang, C., et al., Dental Implants Loaded With Bioactive Agents Promote Osseointegration in Osteoporosis: A Review. Front Bioeng Biotechnol, 2021. 9: p. 591796.
71. Liu, L., et al., The synergistic promotion of osseointegration by nanostructure design and silicon substitution of hydroxyapatite coatings in a diabetic model. J Mater Chem B, 2020. 8(14): p. 2754-2767.
72. Liu, L., et al., Bone marrow stromal cells stimulated by strontium-substituted calcium silicate ceramics: release of exosomal miR-146a regulates osteogenesis and angiogenesis. Acta Biomater, 2021. 119: p. 444-457.
73. Wang, X.-F., et al., Nano hydroxyapatite particles promote osteogenesis in a three dimensional bio-printing construct consisting of alginate/gelatin/hASCs. RSC advances, 2016. 6(8): p. 6832-6842.
74. J, W., Das Gesetz der transformation der Knochen. A. Hirshwald, 1892. 1: p. 1-152.
75. Hu, C. and Q.-H. Qin, Bone remodeling and biological effects of mechanical stimulus. AIMS Bioengineering, 2020. 7(1): p. 12-28.
76. Robling, A. G. and C. H. Turner, Mechanical signaling for bone modeling and remodeling. Critical Reviews™ in Eukaryotic Gene Expression, 2009. 19(4).
77. Staff, P. H., The effects of physical activity on joints, cartilage, tendons and ligaments. Scandinavian journal of social medicine. Supplementum, 1982: p. 59-63.
78. Wang, L., et al., Mechanical regulation of bone remodeling. Bone Research, 2022. 10(1): p. 1-15.
79. Sheikh, Z., et al., Biodegradable Materials for Bone Repair and Tissue Engineering Applications. Materials (Basel), 2015. 8: p. 5744-5794.

1-27. (canceled)

28. An implantable bone anchor comprising:
a first portion for immediate fixation of the bone anchor to a bone upon implantation of the bone anchor, and
a second portion including a swelling material that expands to provide long-term fixation to the bone.

29. The implantable bone anchor of claim 28, wherein the first portion includes threads on an outer surface thereof.

30. The implantable bone anchor of claim 28, wherein the first portion is made of a swelling material that may be the same or different swelling material as included in the second portion.

31. The implantable bone anchor of claim 28 wherein the second portion is an outer sleeve surrounding at least part of the first portion.

32. The implantable bone anchor of claim 28, where in the implantable bone anchor is cylindrical.

33. The implantable bone anchor of claim 28, wherein the swelling material of the second portion is a porous swelling copolymer.

34. The implantable bone anchor of claim 30, wherein the swelling material of the first portion is a porous swelling copolymer.

35. The implantable bone anchor of claim 33, wherein the porous swelling copolymer has an average pore size of from about 50 microns to about 750 microns.

36. The implantable bone anchor of claim 33, wherein the porous swelling copolymer has an average pore size of from about 250 microns to about 700 microns.

37. The implantable bone anchor of claim 33, wherein the porous swelling copolymer has an average pore size of from about 400 microns to about 600 microns.

38. The implantable bone anchor of claim 28, wherein the second portion comprises a solid swelling copolymer and a porous swelling copolymer.

39. The implantable bone anchor of claim 33, wherein the porous swelling copolymer has a porosity of from about 30% to about 70%.

40. The implantable bone anchor of claim 38, wherein the solid swelling copolymer and the porous swelling copolymer each comprise a crosslinked copolymer of a hydrophobic monomer and a hydrophilic monomer.

41. The implantable bone anchor of claim 40, wherein the hydrophobic monomer is selected from the group consisting of methyl methacrylate, styrene, vinyl toluene, carvacryl (meth)acrylate, menthyl (meth)acrylate, thymyl (meth)acrylate, cyclohexyl (meth)acrylate, butyl (meth)acrylate and hexyl (meth)acrylate.

42. The implantable bone anchor of claim 41, wherein the hydrophilic monomer is selected from the group consisting of 2-hydroxy ethyl (meth)acrylate (HEMA or HEA), N,N-dimethyl acrylamide, N-Isopropylacrylamide (NiPAM), 2-dimethylaminoethyl (meth)acrylate (DMAEMA), dimethylaminoethyl acrylate DMAEA), acrylic acid (AA), polyethylene glycol (meth)acrylate (PEG-MA or PEG-A) and glyceryl mono(meth)acrylate.

43. The implantable bone anchor of claim 40, wherein the hydrophobic monomer is methyl methacrylate and the hydrophilic monomer is acrylic acid.

44. The implantable bone anchor of claim 40, wherein one or both of the copolymer(s) are crosslinked using a cross-linking agent selected from the group consisting of allyl methacrylate, hexane diol di(meth)acrylate, butane diol di(meth)acrylate, bisphenol A-glycidyl methacrylate (BisGMA), ethylene glycol di(meth)acrylate and polyethylene glycol di(meth)acrylate.

45. The implantable bone anchor of claim 33, wherein the porous swelling copolymer has a varying porosity with a porosity distribution that decreases from an outer portion of the porous swelling copolymer to an inner portion of the porous swelling copolymer.

46. The implantable bone anchor of claim 45, wherein the outer portion of the porous swelling copolymer has a pore size of from about 50 microns to about 600 microns, and the inner portion of the porous swelling copolymer has a pore size of from about 400 microns to about 750 microns.

47. The implantable bone anchor of claim 45, wherein the outer portion of the porous swelling copolymer has a porosity of from about 40% to about 70%, and the inner portion of the porous swelling copolymer has a porosity in the range of from about 30% to about 60%.

48. The implantable bone anchor of claim 33, wherein porosity is introduced to the porous swelling copolymer by preparing the copolymer with inclusions of one or more dissolvable materials and subsequently leaching at least some of the one or more dissolvable materials out of the porous swelling copolymer.

49. The implantable bone anchor of claim 33, wherein porosity is introduced to the porous swelling copolymer by use of 3D stereolithographic digital light processing.

50. A bone anchor comprising:
a tip;
an anchoring element including:
a distal portion configured for connection to the tip,
a proximal portion including two suture tunnels therein which are adapted for receiving a suture, and
a central portion between the distal and proximal portions including an additional suture tunnel adapted for receiving the suture.

51. The bone anchor of claim 50, further comprising a hollow swelling sleeve including:
a tube of a solid swelling copolymer configured for surrounding the central portion of the anchoring element, and
an outer layer of a porous swelling copolymer surrounding the tube.

52. The bone anchor of claim 50, wherein the proximal portion is provided with circumferential threads on an outer surface thereof.

53. A method of preparing an implantable bone fixation device comprising steps of:
forming a sacrificial template comprising one or more water soluble crystals,
introducing a liquid copolymer into the sacrificial template,
curing the sacrificial template comprising the liquid copolymer to form a polymer crystal composite, and
washing the polymer crystal composite with water to remove the one or more of the water soluble crystals from the polymer crystal composite to form the bone fixation device.

54. The method of claim 53, wherein the one or more water soluble crystals comprises one or more sodium chloride crystals.
